(19) United States
(12) Patent Application Publication
(10) Pub. No.: US 2022/0125760 A1
(43) Pub. Date: Apr. 28, 2022

(54) USE OF USP7 INHIBITORS FOR THE TREATMENT OF ACUTE MYELOID LEUKEMIA (AML)

(71) Applicants: INSERM (Institut National de la Santé et de la Recherche Médicale), Paris (FR); UNIVERSITÉ PAUL SABATIER TOULOUSE III, Toulouse (FR); CENTRE NATIONAL DE LA RECHERCHE SCIENTIFIQUE (CNRS), Paris (FR)

(72) Inventors: Stéphane MANENTI, Toulouse Cedex 1 (FR); Christine DIDIER, Toulouse Cedex 1 (FR); Maëlle CARTEL, Toulouse Cedex 1 (FR)

(21) Appl. No.: 17/430,371

(22) PCT Filed: Feb. 13, 2020
(86) PCT No.: PCT/EP2020/053711

§ 371 Date: Aug. 12, 2021

(30) Foreign Application Priority Data
Feb. 14, 2019 (EP) 19305181.0

(51) Int. Cl. A61K 31/381; A61P 35/02; A61K 31/7068

(52) U.S. Cl. CPC A61K 31/381; A61K 31/7068; A61P 35/02

(57) ABSTRACT

Resistance of acute myeloid leukemia (AML) cells to DNA damaging therapeutic agents is dependent on CHK1 protein levels. Here, the inventors demonstrate that in AML, CHK1 protein stability relies on the expression and activity of Ubiquitin Specific Protease 7 (USP7). CHK1 and USP7 levels are positively correlated in AML cell lines and primary patient specimens with high CHK1 protein levels. USP7 associates with CHK1, leading to its stabilization by deubiquitinylation, and this association is enhanced in response to cytarabine treatment. Pharmacological or RNA interference-mediated inhibition of USP7 significantly reduced AML proliferation in vitro and in vivo, and increased AML cell death. It is important to note that USP7 inhibition synergized with cytarabine to kill AML cell lines. This is also the case in primary patient specimens with high CHK1 levels. Transcriptomic dataset analyses revealed that a USP7 gene signature is highly enriched in cells from AML patients at relapse, as well as in residual blasts from Patient Derived Xenograft (PDX) models treated with clinically relevant doses of cytarabine, strongly suggesting a relationship between USP7 expression and resistance to therapy. Finally, single cell analysis from AML patient at relapse versus diagnosis showed that a gene signature of the pre-existing subpopulation responsible for relapse is enriched in transcriptomes of patients with high USP7 level. Altogether, these data demonstrate that USP7 is a master regulator of CHK1 protein kinase in AML cells, and represents both a marker of resistance to chemotherapeutic treatments, as well as a potential therapeutic target to overcome treatment resistance.

### FIELD OF THE INVENTION

The present invention relates to the use of USP7 inhibitors for the treatment of acute myeloid leukemia (AML).

### BACKGROUND OF THE INVENTION

Acute myeloid leukemia (AML) originates from the transformation and clonal expansion of undifferentiated hematopoietic progenitors, characterized by altered growth, differentiation, and proliferation capacities, which result in failure of bone marrow hematopoietic functions. Although a majority of AML patients initially respond to standard induction therapy, a protocol combining cytarabine (AraC) with an anthracycline, relapses are common and the 5-year overall survival remains very poor.¹ Whole-genome sequencing analyses have highlighted the molecular heterogeneity of AML and allowed risk-based stratification.² For some mutations resulting in oncogenic signaling, specific inhibitors have been developed and included in clinical strategies.^{3,4} Unfortunately, these strategies have shown only transient and limited efficacy due to a variety of resistance mechanisms arising in minor subpopulations of resistant leukemic cells (RLC) that can initiate relapse.⁵ Thus, identifying these resistance mechanisms and new potential drug targets is acutely needed in AML.

We recently reported that CHEK1 expression is an independent prognostic marker in AML. High CHEK1 transcript levels in leukemic cells were associated with increased risk of relapse and poor survival in a cohort of AML patients who had received first-line cytarabine and anthracycline chemotherapy.⁶ Moreover, resistance to cytarabine in primary AML cells correlated with increased abundance of Checkpoint Kinase 1 protein (CHK1), encoded by CHEK1, and pharmacological inhibition of CHK1 restored sensitivity of high CHK1 leukemic cells to cytarabine. These results suggest that a subpopulation of AML cells with high CHK1 levels may survive to selective pressure by cytarabine and form a residual aggressive tumor at the origin of relapse. While we clearly documented the heterogeneity of CHK1 abundance in primary AML samples, the mechanisms by which CHK1 protein level is controlled in these leukemic cells remained unclear. Consequently, a better understanding of the molecular pathways governing CHK1 levels in AML may be of interest to define new therapeutic avenues in these pathologies.

Several transcriptional and post-transcriptional pathways regulating CHK1 protein levels have been described, including mechanisms positively or negatively affecting its stability. Deregulation of the ubiquitylation and deubiquitylation process may underlie the heterogeneity of CHK1 expression observed in AML patient cells. CHK1 is degraded by the ubiquitin-proteasome system in response to genotoxic stress, a mechanism allowing cells to recover from DNA damage and contributing to checkpoint termination.⁷ Ubiquitin ligases involved in CHK1 ubiquitylation and degradation during normal cell cycle progression or in response to DNA damage include CUL4-DDB1-CDT2 and CUL1-SKP1-Fbx6.⁸⁻¹⁰ Inversely, stabilization of CHK1 by the ubiquitin specific proteases USP1, USP3 and USP7 has been described.¹¹⁻¹³ Interestingly, deubiquitylating enzymes, and in particular USP7, were recently pointed as promising therapeutic targets in cancer. Ubiquitin-specific protease 7 (USP7) belongs to a class of cysteine protease deubiquitinating enzymes (DUBs), and plays critical roles in many signaling pathways by deubiquitinating a wide range of targets. USP7 has been involved in the regulation of apoptosis and senescence by modulating the p53 pathway, either directly deubiquitinating p53 or by stabilizing MDM2, an E3-ubiquitin ligase that ubiquitylates and targets p53 for proteasomal degradation.¹⁴⁻¹⁷ Independently of its role on p53, USP7 modulates various pathways both in homeostasis or during oncogenesis by targeting a large panel of substrates.¹⁸⁻²⁹ Consequently, USP7 is at the center of a complex network, and recent efforts have focused on the discovery and development of small molecule inhibitors of this protein.³⁰⁻³⁶ These inhibitors were found to enhance apoptosis in chronic lymphocytic leukemia³⁷ and multiple myeloma³⁸, and to reduce neuroblastoma growth in vivo³⁹.

### SUMMARY OF THE INVENTION

As defined by the claims, the present invention relates to the use of USP7 inhibitors for the treatment of acute myeloid leukemia (AML).

### DETAILED DESCRIPTION OF THE INVENTION

Resistance of acute myeloid leukemia (AML) cells to DNA damaging therapeutic agents is dependent on CHK1 protein levels. Here, the inventors demonstrate that in AML, CHK1 protein stability relies on the expression and activity of Ubiquitin Specific Protease 7 (USP7). CHK1 and USP7 levels are positively correlated in AML cell lines and primary patient specimens with high CHK1 protein levels. USP7 associates with CHK1, leading to its stabilization by deubiquitinylation, and this association is enhanced in response to cytarabine treatment. Pharmacological or RNA interference-mediated inhibition of USP7 significantly reduced AML proliferation in vitro and in vivo, and increased AML cell death. It is important to note that USP7 inhibition synergized with cytarabine to kill AML cell lines. This is also the case in primary patient specimens with high CHK1 levels. Transcriptomic dataset analyses revealed that a USP7 gene signature is highly enriched in cells from AML patients at relapse, as well as in residual blasts from Patient Derived Xenograft (PDX) models treated with clinically relevant doses of cytarabine, strongly suggesting a relationship between USP7 expression and resistance to therapy. Finally, single cell analysis from AML patient at relapse versus diagnosis showed that a gene signature of the pre-existing subpopulation responsible for relapse is enriched in transcriptomes of patients with high USP7 level. Altogether, these data demonstrate that USP7 is a master regulator of CHK1 protein kinase in AML cells, and represents both a marker of resistance to chemotherapeutic treatments, as well as a potential therapeutic target to overcome treatment resistance.

Accordingly, the first object of the present invention relates to a method of treating acute myeloid leukemia (AML) in a patient in need thereof comprising administering to the patient a therapeutically effective amount of a USP7 inhibitor.

As used herein, the term “acute myeloid leukemia” or “AML”, also known as “acute myelogenous leukemia”, has its general meaning in the art and refers to a cancer of the myeloid line of blood cells, characterized by the rapid growth of abnormal white blood cells that accumulate in the bone marrow and interfere with the production of normal blood cells. AML may be classified using either the World Health Organization classification (Vardiman J W, Harris N L, Brunning R D (2002). “The World Health Organization (WHO) classification of the myeloid neoplasms”. Blood 100 (7): 2292-302); or the FAB classification (Bennett J, Catovsky D, Daniel M, Flandrin G, Galton D, Gralnick H, Sultan C (1976). “Proposals for the classification of the acute leukaemias. French-American-British (FAB) co-operative group”. Br J Haematol 33 (4): 451-8.).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

A further object of the present invention relates to a method of treating chemoresistant acute myeloid leukemia (AML) in patient in need thereof comprising administering to the patient a therapeutically effective amount of a USP7 inhibitor.

As used herein; the term “chemoresistant acute myeloid leukemia” refers to the clinical situation in a patient suffering from acute myeloid leukemia when the proliferation of leukemic cells cannot be prevented or inhibited by means of a chemotherapeutic agent or a combination of chemotherapeutic agents usually used to treat AML, at an acceptable dose to the patient.

As used herein, the term “chemotherapeutic agent” refers to any chemical agent with therapeutic usefulness in the treatment of cancer. Chemotherapeutic agents as used herein encompass both chemical and biological agents. These agents function to inhibit a cellular activity upon which the leukemic cell depends for continued survival. Categories of chemotherapeutic agents include alkylating/alkaloid agents, antimetabolites, hormones or hormone analogues, and miscellaneous antineoplastic drugs. Most if not all of these drugs are directly toxic to leukemic cells and do not require immune stimulation. Suitable chemotherapeutic agents are described, for example, in Slapak and Kufe, Principles of Cancer Therapy, Chapter 86 in Harrison's Principles of Internal medicine, 14th edition; Perry et al, Chemotherapeutic, Ch 17 in Abeloff, Clinical Oncology 2nd ed., 2000 ChrchillLivingstone, Inc.; Baltzer L. and Berkery R. (eds): Oncology Pocket Guide to Chemotherapeutic, 2nd ed. St. Louis, mosby-Year Book, 1995; Fischer D. S., Knobf M. F., Durivage H J. (eds): The Cancer Chemotherapeutic Handbook, 4th ed. St. Louis, Mosby-Year Handbook.

In some embodiments the chemotherapeutic agent is cytarabine (cytosine arabinoside, Ara-C, Cytosar-U), quizartinib (AC220), sorafenib (BAY 43-9006), lestaurtinib (CEP-701), midostaurin (PKC412), carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab (Rituxan®), gemtuzumab ozogamicin, imatinib mesylate, Cytosar-U), melphalan, busulfan (Myleran®), thiotepa, bleomycin, platinum (cisplatin), cyclophosphamide, (Cytoxan®), daunorubicin, doxorubicin, idarubicin, mitoxantrone, 5-azacytidine, cladribine, fludarabine, hydroxyurea, 6-mercaptopurine, methotrexate, 6-thioguanine, or any combination thereof. In some embodiments, the leukemia is resistant to a combination of daunorubicin, or idarubicin plus cytarabine (AraC).

In some embodiments, the chemotherapeutic agent is a BCL2 inhibitor. In some embodiments, the Bc1-2 inhibitor comprises 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide (also known as, and optionally referred to herein as, venetoclax, or ABT-199, or GDC-0199) or a pharmaceutically acceptable salt thereof.

In some embodiments, the chemotherapeutic agent is a FLT3 inhibitor. Examples of FLT3 inhibitors include N-(2-diethylaminoethyl)-5-[(Z)-(5-fluoro-2-oxo-1H-indol-3-ylidene)methyl]-2,4-dimethyl-1H-pyrrole-3-carboxamide, sunitinib, also knows as SU11248, and marketed as SUTENT (sunitinib malate); 4-[4-[[4-chloro-3-(trifluoromethyl)phenyl]carbamoylamino]phenoxy]-N-methyl-pyridine-2-carboxamide, sorafenib, also known as BAY 43-9006, marketed as NEXAVAR (sorafenib); (9S,10R,11R,13R)-2,3,10,11,12,13-Hexahydro-10-methoxy-9-methyl-11-(methylamino)-9,13-epoxy-1H,9H-diindolo [1,2,3-gh:3′,2′,1′-lm]pyrrolo[3,4-j][1,7]benzodiamzonine-1-one, also known as midostaurin or PKC412; (5 S,6 S,8R)-6-Hydroxy-6-(hydroxymethyl)-5-methyl-7,8,14,15-tetrahydro-5H-16-oxa-4b,8a,14-triaza-5, 8-methanodibenzo[b,h]cycloocta[jkl]cyclopenta[e]-as-indacen-13 (6H)-one, also known as lestaurtinib or CEP-701; 1-(5-(tert-Butyl)isoxazol-3-yl)-3-(4-(7-(2-morpholinoethoxy)benzo[d]imidazo[2,1-b]thiazol-2-yl)phenyl)urea, also known as Quizartinib or AC220; 1-(2-{5-[(3-Methyloxetan-3-yl)methoxy]-1H-benzimidazol-1-yl}quinolin-8-yl)piperidin-4-amine, also known as Crenolanib or CP-868,596-26. See, e.g., Wander S. A., TherAdv Hematol. 5: 65-77 (2014). Other FLT3 inhibitors include Pexidartinib (PLX-3397), Tap et al, N Engl J Med, 373:428-437 (2015); gilteritinib (ASP2215), Smith et al., Blood: 126 (23) (2015); FLX-925, also known as AMG-925, Li et al. Mol. Cancer Ther. 14: 375-83 (2015); and G-749, Lee et al, Blood. 123: 2209-2219 (2014).

In some embodiments, the chemotherapeutic agent is an IDH (isocitrate dehydrogenase) inhibitor. In some embodiments, the IDH inhibitor is a member of the oxazolidinone (3-pyrimidinyl-4-yl-oxazolidin-2-one) family, and is a specific inhibitor of the neomorphic activity of IDH1 mutants and has the chemical name (S)-4-isopropyl-3-(2-(((S)-1-(4 phenoxyphenyl)ethyl)amino)pyrimidin-4-yl)oxazolidin-2-one.

A further object of the present invention relates to a method of treating acute myeloid leukemia (AML) in a patient in need thereof comprising administering to the patient a therapeutically effective combination comprising at least one chemotherapeutic agent and a USP7 inhibitor.

As used herein, the term “combination” is intended to refer to all forms of administration that provide a first drug together with a further (second, third . . . ) drug. The drugs may be administered simultaneous, separate or sequential and in any order. Drugs administered in combination have biological activity in the patient to which the drugs are delivered. Within the context of the invention, a combination thus comprises at least two different drugs, and wherein one drug is at least a chemotherapeutic agent (e.g. cytarabine) and wherein the other drug is at least a USP7 inhibitor. In some instance, the combination of the present invention results in the synthetic lethality of the leukemic cells.

A further object of the present invention relates to a method for enhancing the potency of a chemotherapeutic agent administered to a patient suffering from AML as part of a treatment regimen, the method comprising administering to the patient a pharmaceutically effective amount of a USP7 inhibitor in combination with at least one chemotherapeutic agent.

Thus the expression “enhancing the potency of a chemotherapeutic agent” refers to the ability of the USP7 inhibitor to increase the ability of the chemotherapeutic agent to kill tumor cells by more than about 20%, preferably with at least about 30%, at least about 40%, at least about 50%, at least about 100%.

A further object of the present invention relates to a method of preventing relapse in a patient suffering from AML who was treated with chemotherapy comprising administering to the patient a therapeutically effective amount of a USP7 inhibitor.

As used herein, the term “relapse” refers to the return of cancer after a period of improvement in which no cancer could be detected. Thus, the method of the present invention is particularly useful to prevent relapse after putatively successful treatment with chemotherapy.

As used herein, the term “USP7” has its general meaning in the art and refers to the ubiquitin-specific protease 7. USP7 is an Ubiquitin Specific Protease (USP) family deubiquitinase (DUB) that was originally identified as an enzyme that interacted with virally-encoded proteins of the Herpes simplex virus and later the Epstein-Barr virus. (Everett R. D., Meredith M, Orr A., Cross A, Kathoria M, Parkinson J. “A novel ubiquitin-specific protease is dynamically associated with the PML nuclear domain and binds to a herpes virus regulatory protein,” EMBO J. 16(7):1519-30 (1997); Holowaty M. N., Zeghouf M., Wu H., et al. “Protein profiling with Epstein-Barr nuclear antigen-1 reveals an interaction with the herpesvirus-associated ubiquitin-specific protease HAUSP/USP7,” J. Biol. Chem. 278(32):29987-94 (2003)) Ubiquitin Specific Proteases (USPs) specifically cleave the isopeptide bond at the carboxy terminus of ubiquitin. In contrast to other DUB classes, which are thought to generally regulate ubiquitin homeostasis or to be involved in pre-processing of linear ubiquitin chains, USPs remove ubiquitin from specific targets. Given this substrate specificity combined with the numerous roles ubiquitination has in the cell, USPs are important regulators of a multitude of pathways, ranging from preventing the proteolysis of ubquitinated substrates, to controlling their nuclear localization.

As used herein, the term “USP7 inhibitor” refers to a molecule which suppresses the expression of USP7 protein (i.e. interferes with expression of the USP7 gene), including suppression of transcription or translation, and/or a molecule that directly inhibits USP7 activity, for example by binding to the USP7 protein (USP7 inhibitor ligand). Such inhibitors may comprise any of the group comprising of inhibitors of expression as disclosed herein (e.g. siRNA, miRNA, shRNA, antisense oligonucleotides, or ribozymes), peptides, ligands, chemical inhibitors (i.e. small molecule), antibodies and antibody fragments.

USP7 inhibitors are well known in the art and are typically described in:
Desroses M, Altun M The Next Step Forward in Ubiquitin-Specific Protease 7 Selective Inhibition. Cell Chem Biol. 2017 Dec. 21; 24(12): 1429-1431.
Gavory G, O'Dowd C R, Helm M D, Flasz J, Arkoudis E, Dossang A, Hughes C, Cassidy E, McClelland K, Odrzywol E, Page N, Barker O, Miel H, Harrison T Discovery and characterization of highly potent and selective allosteric USP7 inhibitors. Nat Chem Biol. 2018 February; 14(2): 118-125.
Jing B, Liu M, Yang L, Cai H Y, Chen J B, Li Z X Kou X, Wu Y Z, Qin D J, Zhou L, Jin J, Lei H, Xu H Z, Wang W W, Wu Y L. Characterization of naturally occurring pentacyclic triterpenes as novel inhibitors of deubiquitinating protease USP7 with anticancer activity in vitro. Acta Pharmacol Sin. 2018 March; 39(3):492-498.
Di Lello P, Pastor R, Murray J M, Blake R A, Cohen F, Crawford T D, Drobnick J, Drummond J, Kategaya L, Kleinheinz T, Maurer T, Rougé L, Zhao X Wertz I, Ndubaku C, Tsui V. Discovery of Small-Molecule Inhibitors of Ubiquitin Specific Protease 7 (USP7) Using Integrated NMR and in Silico Techniques. J Med Chem. 2017 Dec. 28; 60(24): 10056-10070.
Zhou J, Wang J, Chen C, Yuan H, Wen X, Sun H. USP7: Target Validation and Drug Discovery for Cancer Therapy. Med Chem. 2018; 14(1):3-18.
Lamberto I, Liu X Seo H S, Schauer N J, Jacob R E, Hu W, Das D, Mikhailova T, Weisberg E L, Engen J R, Anderson K C, Chauhan D, Dhe-Paganon S, Buhrlage Si Structure-Guided Development of a Potent and Selective Non-covalent Active-Site Inhibitor of USP7. Cell Chem Biol. 2017 Dec. 21; 24(12): 1490-1500.e11.
Pozhidaeva A, Valles G, Wang F, Wu J, Sterner D E, Nguyen P, Weinstock J, Kumar KGS, Kanyo J, Wright D, Bersonova I. USP7-Specific Inhibitors Target and Modify the Enzyme's Active Site via Distinct Chemical Mechanisms. Cell Chem Biol. 2017 Dec. 21; 24(12):1501-1512.e5.
Turnbull A P, Ioannidis S, Krajewski W W, Pinto-Fernandez A, Heride C, Martin A C L, Tonkin L M, Townsend E C, Buker S M, Lancia D R, Caravella J A, Toms A V, Charlton 7M, Landenranta J, Wilker E, Follows B C, Evans N J, Stead L, Alli C, Zarayskiy V V, Talbot A C, Buckmelter A J, Wang M, McKinnon C L, Saab F, McGouran J F, Century H, Gersch M, Pittman M S, Marshall C G, Raynham 7M, Simcox M, Stewart LMD, McLoughlin S B, Escobedo J A, Bair K W, Dinsmore C J, Hammonds T R, Kim S, Urbe S, Clague M J, Kessler B M, Komander D. Molecular basis of USP7 inhibition by selective small-molecule inhibitors. Nature. 2017 Oct. 26; 550(7677):481-486.
Kategaya L, Di Lello P, Rouge L, Pastor R, Clark K R, Drummond J, Kleinheinz T, Lin E, Upton J P, Prakash S, Heideker J, McCleland M, Ritorto M S, Alessi D R, Trost M, Bainbridge T W, Kwok M C M, Ma T P, Stiffler Z, Brasher B, Tang Y, Jaishankar P, Hearn B R, Renslo A R, Arkin M R, Cohen F, Yu K, Peale F, Gnad F, Chang M T, Klijn C, Blackwood E, Martin S E, Forrest W F, Ernst J A, Ndubaku C, Wang X Beresini M H, Tsui V, Schwerdtfeger C, Blake R A, Murray J, Maurer T, Wertz I E. USP7 small-molecule inhibitors interfere with ubiquitin binding. Nature. 2017 Oct. 26; 550(7677):534-538.

In some embodiments, the USP7 inhibitor is selected from quinazolinones and azaquinazolinones as described in U.S. Pat. No. 9,840,491.

In some embodiments, the USP7 inhibitor is selected from pyrrolo and pyrazolopyrimidines as described in U.S. Pat. No. 9,902,728.

In some embodiments, the USP7 inhibitor is selected from thienopyrimidinones as described in U.S. Pat. No. 9,932,351.

In some embodiments, the USP7 inhibitor is selected from Isothiazolopyrimidinones, pyrazolopyrimidinones, and pyrrolopyrimidinones as described in U.S. Pat. No. 9,938,300.

In some embodiments, the USP7 inhibitor is selected from pyrrolotriazinones and imidazotriazinones as described in U.S. Pat. No. 10,000,495.

In some embodiments, the USP7 inhibitor is selected from compounds described in WO2016109480. In particular, the compound is selected from the group consisting of:
3-((4-hydroxy-1-(3-phenylbutanoyl)piperidin-4-yl)methyl)pyrrolo[2,1-f][1,2,4]triazin-4(3H)-one;
3-((1-([1,1′-biphenyl]-2-carbonyl)-4-hydroxypiperidin-4-yl)methyl)pyrrolo[2,1-f][1,2,4]triazin-4(3H)-one;
3-((4-hydroxy-1-(2-(thiophen-3-yl)benzoyl)piperidin-4-yl)methyl)pyrrolo[2,1-f][1,2,4]triazin-4(3H)-one;
3-((1-(3′-fluoro-[1,1′-biphenyl]-2-carbonyl)-4-hydroxypiperidin-4-yl)methyl)pyrrolo[2,1-f][1,2,4]triazin-4(3H)-one;
3-((1-(3-(benzo[d][1,3]dioxol-5-yl)benzoyl)-4-hydroxypiperidin-4-yl)methyl)pyrrolo[2,1-f][1,2,4]triazin-4(3H)-one;
3-((4-hydroxy-1-(2′-methyl-[1,1′-biphenyl]-3-carbonyl)piperidin-4-yl)methyl)pyrrolo[2,1-f][1,2,4]triazin-4(3H)-one;
3-((4-hydroxy-1-(4′-methyl-[1,1′-biphenyl]-3-carbonyl)piperidin-4-yl)methyl)pyrrolo[2,1-f][1,2,4]triazin-4(3H)-one;
3-((4-hydroxy-1-(4′-methoxy-[1,1′-biphenyl]-3-carbonyl)piperidin-4-yl)methyl)pyrrolo[2,1-f][1,2,4]triazin-4(3H)-one;
3-((1-(4′-fluoro-3′-methyl-[1,1′-biphenyl]-3-carbonyl)-4-hydroxypiperidin-4-yl)methyl)pyrrolo[2,1-f][1,2,4]triazin-4(3H)-one;
3-((4-hydroxy-1-(3′-methoxy-[1,1′-biphenyl]-3-carbonyl)piperidin-4-yl)methyl)pyrrolo[2,1-f][1,2,4]triazin-4(3H)-one;
3-((1-([1,1′-biphenyl]-3-carbonyl)-4-hydroxypiperidin-4-yl)methyl)pyrrolo[2,1-f][1,2,4]triazin-4(3H)-one;
3-((1-(4′-chloro-[1,1′-biphenyl]-3-carbonyl)-4-hydroxypiperidin-4-yl)methyl)pyrrolo[2,1-f][1,2,4]triazin-4(3H)-one;
3-((1-(3′-chloro-[1,1′-biphenyl]-3-carbonyl)-4-hydroxypiperidin-4-yl)methyl)pyrrolo[2,1-f][1,2,4]triazin-4(3H)-one;
3-((4-hydroxy-1-(4′-isopropyl-[1,1′-biphenyl]-3-carbonyl)piperidin-4-yl)methyl)pyrrolo[2,1-f][1,2,4]triazin-4(3H)-one;
3-((4-hydroxy-1-(4′-(trifluoromethyl)-[1,1′-biphenyl]-3-carbonyl)piperidin-4-yl)methyl)pyrrolo[2,1-f][1,2,4]triazin-4(3H)-one;
3′-(4-hydroxy-4-((4-oxopyrrolo[2,1-f][1,2,4]triazin-3 (4H)-yl)methyl)piperidine-1-carbonyl)-[1,1′-biphenyl]-4-carboxamide;
3-((1-(3′,4′-dimethyl-[1,1′-biphenyl]-3-carbonyl)-4-hydroxypiperidin-4-yl)methyl)pyrrolo[2,1-f][1,2,4]triazin-4(3H)-one;
3′-(4-hydroxy-4-((4-oxopyrrolo[2,1-f][1,2,4]triazin-3 (4H)-yl)methyl)piperidine-1-carbonyl)-[1,1′-biphenyl]-2-carbonitrile;
3′-(4-hydroxy-4-((4-oxopyrrolo[2,1-f][1,2,4]triazin-3 (4H)-yl)methyl)piperidine-1-carbonyl)-[1,1′-biphenyl]-4-carbonitrile;
3-((4-hydroxy-1-(3-(5-methylthiophen-2-yl)benzoyl)piperidin-4-yl)methyl)pyrrolo[2,1-f][1,2,4]triazin-4(3H)-one;
3-((4-hydroxy-1-(3-(quinolin-6-yl)benzoyl)piperidin-4-yl)methyl)pyrrolo[2,1-f][1,2,4]triazin-4(3H)-one;
3-((4-hydroxy-1-(3-(imidazo[1,2-a]pyridin-6-yl)benzoyl)piperidin-4-yl)methyl)pyrrolo[2,1-f][1,2,4]triazin-4(3H)-one;
3-((1-(3-(benzo[d]thiazol-5-yl)benzoyl)-4-hydroxypiperidin-4-yl)methyl)pyrrolo[2,1-f][1,2,4]triazin-4(3H)-one;
3-((4-hydroxy-1-(3-(5-methyl-1H-indazol-4-yl)benzoyl)piperidin-4-yl)methyl)pyrrolo[2,1-f][1,2,4]triazin-4(3H)-one;
3-((4-hydroxy-1-(3-(1-methyl-1H-indol-2-yl)benzoyl)piperidin-4-yl)methyl)pyrrolo[2,1-f][1,2,4]triazin-4(3H)-one;
N-cyclopentyl-3′-(4-hydroxy-4-((4-oxopyrrolo[2,1-f][1,2,4]triazin-3 (4H)-yl)methyl)piperidine-1-carbonyl)-[1,1′-biphenyl]-3-carboxamide;
3-((4-hydroxy-1-(3-(thiophen-2-yl)benzoyl)piperidin-4-yl)methyl)pyrrolo[2,1-f][1,2,4]triazin-4(3H)-one;
3-((4-hydroxy-1-(3-(thiophen-3-yl)benzoyl)piperidin-4-yl)methyl)pyrrolo[2,1-f][1,2,4]triazin-4(3H)-one;
3-((142′-fluoro-[1,1′-biphenyl]-3-carbonyl)-4-hydroxypiperidin-4-yl)methyl)pyrrolo[2,1-f][1,2,4]triazin-4(3H)-one;
3-((1-(3′-fluoro-[1,1′-biphenyl]-3-carbonyl)-4-hydroxypiperidin-4-yl)methyl)pyrrolo[2,1-f][1,2,4]triazin-4(3H)-one;
2-(4-(4-hydroxy-4-((4-oxopyrrolo[2,1-f][1,2,4]triazin-3 (4H)-yl)methyl)piperidine-1-carbonyl)phenyl)-2-methylpropanenitrile;
3-((4-hydroxy-1-(2-phenyloxazole-5-carbonyl)piperidin-4-yl)methyl)pyrrolo[2,1-f][1,2,4]triazin-4(3H)-one;
3-((1-(1-(benzo[d]oxazol-2-yl)piperidine-4-carbonyl)-4-hydroxypiperidin-4-yl)methyl)pyrrolo[2,1-f][1,2,4]triazin-4(3H)-one;
3-((1-(3-(1H-pyrazol-1-yl)butanoyl)-4-hydroxypiperidin-4-yl)methyl)pyrrolo[2,1-f][1,2,4]triazin-4(3H)-one;
3-((4-hydroxy-1-(3′-methoxy-[1,1′-biphenyl]-4-carbonyl)piperidin-4-yl)methyl)pyrrolo[2,1-f][1,2,4]triazin-4(3H)-one;
3-((1-([1,1′-biphenyl]-4-carbonyl)-4-hydroxypiperidin-4-yl)methyl)pyrrolo[2,1-f][1,2,4]triazin-4(3H)-one;
3-((1-(3′-ethoxy-[1,1′-biphenyl]-4-carbonyl)-4-hydroxypiperidin-4-yl)methyl)pyrrolo[2,1-f][1,2,4]triazin-4(3H)-one;
4′-(4-hydroxy-4-((4-oxopyrrolo[2,1-f][1,2,4]triazin-3 (4H)-yl)methyl)piperidine-1-carbonyl)-N,N-dimethyl-[1,1′-biphenyl]-4-carboxamide;
3-((4-hydroxy-1-(4′-(pyrrolidine-1-carbonyl)-[1,1′-biphenyl]-4-carbonyl)piperidin-4-yl)methyl)pyrrolo[2,1-f][1,2,4]triazin-4(3H)-one;
3-((142′,5′-dimethoxy-[1,1′-biphenyl]-4-carbonyl)-4-hydroxypiperidin-4-yl)methyl)pyrrolo[2,1-f][1,2,4]triazin-4(3H)-one;
N-ethyl-4′-(4-hydroxy-4-((4-oxopyrrolo[2,1-f][1,2,4]triazin-3 (4H)-yl)methyl)piperidine-1-carbonyl)-[1,1′-biphenyl]-4-carboxamide;
3-((4-hydroxy-1-(4-(quinolin-3-yl)benzoyl)piperidin-4-yl)methyl)pyrrolo[2,1-f][1,2,4]triazin-4(3H)-one;
3-((4-hydroxy-1-(4-(quinolin-6-yl)benzoyl)piperidin-4-yl)methyl)pyrrolo[2,1-f][1,2,4]triazin-4(3H)-one;
3-((1-(3′,5′-dimethoxy-[1,1′-biphenyl]-4-carbonyl)-4-hydroxypiperidin-4-yl)methyl)pyrrolo[2,1-f][1,2,4]triazin-4(3H)-one;
3-((4-hydroxy-1-(4-(2-methyl quinolin-6-yl)benzoyl)piperidin-4-yl)methyl)pyrrolo[2,1-f][1,2,4]triazin-4(3H)-one;
3-((4-hydroxy-1-(4-(1-methyl-1H-benzo[d]imidazol-5-yl)benzoyl)piperidin-4-yl)methyl)pyrrolo[2,1-f][1,2,4]triazin-4(3H)-one;
3-((1-(4-(benzo[d]oxazol-5-yl)benzoyl)-4-hydroxypiperidin-4-yl)methyl)pyrrolo[2,1-f][1,2,4]triazin-4(3H)-one;
3-((1-(4-([1,2,4]triazolo[1,5-a]pyridin-6-yl)benzoyl)-4-hydroxypiperidin-4-yl)methyl)pyrrolo[2,1-f][1,2,4]triazin-4(3H)-one;
6-chloro-3-((4-hydroxy-1-(3-phenylbutanoyl)piperidin-4-yl)methyl)pyrrol o [2,1-f][1,2,4]triazin-4(3H)-one;
3-((1-(3-chloro-[1,1′-biphenyl]-4-carbonyl)-4-hydroxypiperidin-4-yl)methyl)pyrrolo[2,1-f][1,2,4]triazin-4(3H)-one;
3-((1-(3-chloro-4′-(pyrrolidine-1-carbonyl)-[1,1′-biphenyl]-4-carbonyl)-4-hydroxypiperidin-4-yl)methyl)pyrrolo[2,1-f][1,2,4]triazin-4(3H)-one;
3-((4-hydroxy-1-(4′-(piperidine-1-carbonyl)-[1,1′-biphenyl]-4-carbonyl)piperidin-4-yl)methyl)pyrrolo[2,1-f][1,2,4]triazin-4(3H)-one;
3-((1-(2-benzyl-3,3-dimethylbutanoyl)-4-hydroxypiperidin-4-yl)methyl)pyrrolo[2,1-f][1,2,4]triazin-4(3H)-one;
2-benzyl-3-(4-hydroxy-4-((4-oxopyrrolo[2,1-f][1,2,4]triazin-3 (4H)-yl)methyl)piperidin-1-yl)-3-oxopropanenitrile;
3-((4-hydroxy-1-(4-(2-phenylpropan-2-yl)benzoyl)piperidin-4-yl)methyl)pyrrolo[2,1-f][1,2,4]triazin-4(3H)-one;
(R)-3-((4-hydroxy-1-(3-phenylbutanoyl)piperidin-4-yl)methyl)pyrrolo[2,1-f][1,2,4]triazin-4(3H)-one;
3-((1-(3-(1H-pyrrol-1-yl)butanoyl)-4-hydroxypiperidin-4-yl)methyl)pyrrolo[2,1-f][1,2,4]triazin-4(3H)-one;
3-((4-hydroxy-1-(2-(1,2,3,4-tetrahydronaphthalen-2-yl)acetyl)piperidin-4-yl)methyl)pyrrolo[2,1-f][1,2,4]triazin-4(3H)-one;
3-((4-hydroxy-1-(4-(thiazol-4-yl)benzoyl)piperidin-4-yl)methyl)pyrrol o [2,1-f][1,2,4]triazin-4(3H)-one;
3-((1-(2-benzylbutanoyl)-4-hydroxypiperidin-4-yl)methyl)pyrrolo[2,1-f][1,2,4]triazin-4(3H)-one;
3-((4-hydroxy-1-(4-(phenyl sulfonyl)benzoyl)piperidin-4-yl)methyl)pyrrol o [2,1-f][1,2,4]triazin-4(3H)-one;
3-((4-hydroxy-1-(4-(phenylthi o)benzoyl)piperidin-4-yl)methyl)pyrrolo[2,1-f][1,2,4]triazin-4(3H)-one;
3-((4-hydroxy-1-(2-methyl-3-phenylpropanoyl)piperidin-4-yl)methyl)pyrrolo[2,1-f][1,2,4]triazin-4(3H)-one;
3-((1-(4-((1H-benzo[d]imidazol-1-yl)methyl)benzoyl)-4-hydroxypiperidin-4-yl)methyl)pyrrolo[2,1-f][1,2,4]triazin-4(3H)-one;
3-((1-(4-((1H-pyrazol-1-yl)methyl)benzoyl)-4-hydroxypiperidin-4-yl)methyl)pyrrolo[2,1-f][1,2,4]triazin-4(3H)-one;
3-((4-hydroxy-1-(4-((5-methyl-1H-tetrazol-1-yl)methyl)benzoyl)piperidin-4-yl)methyl)pyrrolo[2,1-f][1,2,4]triazin-4(3H)-one;
3-((4-hydroxy-1-(4-((5-methyl-3-(trifluoromethyl)-1H-pyrazol-1-yl)methyl)benzoyl)piperidin-4-yl)methyl)pyrrolo[2,1-f][1,2,4]triazin-4(3H)-one;
3-((1-(4-((1H-benzo[d][1,2,3]triazol-1-yl)methyl)benzoyl)-4-hydroxypiperidin-4-yl)methyl)pyrrolo[2,1-f][1,2,4]triazin-4(3H)-one;
3-((1-(4-((3,5-dimethyl-1H-pyrazol-1-yl)methyl)benzoyl)-4-hydroxypiperidin-4-yl)methyl)pyrrolo[2,1-f][1,2,4]triazin-4(3H)-one;
3-((4-hydroxy-1-(4-(thiophen-2-ylmethyl)benzoyl)piperidin-4-yl)methyl)pyrrolo[2,1-f][1,2,4]triazin-4(3H)-one;
3-((1-(4-benzoylbenzoyl)-4-hydroxypiperidin-4-yl)methyl)pyrrolo[2,1-f][1,2,4]triazin-4(3H)-one;
3-((1-(4-(ethyl(phenyl)amino)benzoyl)-4-hydroxypiperidin-4-yl)methyl)pyrrolo[2,1-f][1,2,4]triazin-4(3H)-one;
4-(4-(4-hydroxy-4-((4-oxopyrrolo[2,1-f][1,2,4]triazin-3 (4H)-yl)methyl)piperidine-1-carbonyl)piperazin-1-yl)benzonitrile;
3-((4-hydroxy-1-(4-(4-(methyl sulfonyl)phenyl)piperazine-1-carbonyl)piperidin-4-yl)methyl)pyrrolo[2,1-f][1,2,4]triazin-4(3H)-one;
3-((1-(2-chloro-4-(piperidin-1-ylmethyl)benzoyl)-4-hydroxypiperidin-4-yl)methyl)pyrrolo[2,1-f][1,2,4]triazin-4(3H)-one;
3-((4-hydroxy-1-(3-phenylbutanoyl)piperidin-4-yl)methyl)-7-methylpyrrolo[2,1-f][1,2,4]triazin-4(3H)-one;
3-((4-hydroxy-1-(2-methyl-3-phenylpropanoyl)piperidin-4-yl)methyl)-7-methylpyrrolo[2,1-f][1,2,4]triazin-4(3H)-one;
3′-chloro-4′-(4-hydroxy-4-((4-oxopyrrolo[2,1-f][1,2,4]triazin-3 (4H)-yl)methyl)piperidine-1-carbonyl)-N,N-dimethyl-[1,1′-biphenyl]-4-carboxamide;
3-((1-(3-chloro-4′-(piperidine-1-carbonyl)-[1,1′-biphenyl]-4-carbonyl)-4-hydroxypiperidin-4-yl)methyl)pyrrolo[2,1-f][1,2,4]triazin-4(3H)-one;
3-((4-hydroxy-1-(4-phenoxybenzoyl)piperidin-4-yl)methyl)pyrrol o [2,1-f][1,2,4]triazin-4(3H)-one;
(R) N-(3-((4-hydroxy-1-(3-phenylbutanoyl)piperidin-4-yl)methyl)-4-oxo-3,4-dihydropyrrolo[2,1-f][1,2,4]triazin-6-yl)acetamide;
(R)-1-(3-((4-hydroxy-1-(3-phenylbutanoyl)piperidin-4-yl)methyl)-4-oxo-3,4-dihydropyrrolo[2,1-f][1,2,4]triazin-6-yl)-3-methylurea;
3-((4-hydroxy-1-(3-phenylbutanoyl)piperidin-4-yl)methyl)-6-methylpyrrolo[2,1-f][1,2,4]triazin-4(3H)-one;
3-((4-hydroxy-1-(2-methyl-3-phenylpropanoyl)piperidin-4-yl)methyl)-6-methylpyrrolo[2,1-f][1,2,4]triazin-4(3H)-one;
1-(3-chlorophenyl)-3-(3-((1-(cyclopropanecarbonyl)-4-hydroxypiperidin-4-yl)methyl)-4-oxo-3,4-dihydropyrrolo[2,1-f][1,2,4]triazin-6-yl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(m-tolyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(4-fluorobenzyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(4-fluorophenyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(p-tolyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(2,3-dihydro-1H-inden-5-yl)urea;
1-(4-chlorobenzyl)-3-(3-((1-(cyclopropanecarbonyl)-4-hydroxypiperidin-4-yl)methyl)-4-oxo-3,4-dihydropyrrolo[2,1-f][1,2,4]triazin-6-yl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(4-methylbenzyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(3,5-difluorophenyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(2-fluorobenzyl)urea;
(R)-1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(1-phenyl ethyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(3-fluorophenyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(3-fluoro-2-methylphenyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(2,3-dimethylphenyl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(2,4-dimethylphenyl)urea;
1-(4-cyanophenyl)-3-(3-((1-(cyclopropanecarbonyl)-4-hydroxypiperidin-4-yl)methyl)-4-oxo-3,4-dihydropyrrolo[2,1-f][1,2,4]triazin-6-yl)urea;
1-(3-((1-(cyclopropanecarbonyl)-4-hydroxypiperidin-4-yl)methyl)-4-oxo-3,4-dihydropyrrolo[2,1-f][1,2,4]triazin-6-yl)-3-(3-methoxyphenyl)urea;
N-(3-((1-(cyclopropanecarbonyl)-4-hydroxypiperidin-4-yl)methyl)-4-oxo-3,4-dihydropyrrolo[2,1-f][1,2,4]triazin-6-yl)-4-(trifluoromethyl)benzamide;
N-(3-((1-(cyclopropanecarbonyl)-4-hydroxypiperidin-4-yl)methyl)-4-oxo-3,4-dihydropyrrolo[2,1-f][1,2,4]triazin-6-yl)-2-(2,6-dichlorophenyl)acetamide;
N-(3-((1-(cyclopropanecarbonyl)-4-hydroxypiperidin-4-yl)methyl)-4-oxo-3,4-dihydropyrrolo[2,1-f][1,2,4]triazin-6-yl)-2-(4-(trifluoromethoxy)phenoxy)acetamide;
N-(3-((1-(cyclopropanecarbonyl)-4-hydroxypiperidin-4-yl)methyl)-4-oxo-3,4-dihydropyrrolo[2,1-f][1,2,4]triazin-6-yl)-2-(3,4-dichlorophenoxy)acetamide;
N-(3-((1-(cyclopropanecarbonyl)-4-hydroxypiperidin-4-yl)methyl)-4-oxo-3,4-dihydropyrrolo[2,1-f][1,2,4]triazin-6-yl)-2-(2,3-dichlorophenoxy)acetamide;
N-(3-((1-(cyclopropanecarbonyl)-4-hydroxypiperidin-4-yl)methyl)-4-oxo-3,4-dihydropyrrolo[2,1-f][1,2,4]triazin-6-yl)-3,4-dimethylbenzamide;
3-chloro-N-(3-((1-(cyclopropanecarbonyl)-4-hydroxypiperidin-4-yl)methyl)-4-oxo-3,4-dihydropyrrolo[2,1-f][1,2,4]triazin-6-yl)-4-methylbenzamide;
N-(3-((1-(cyclopropanecarbonyl)-4-hydroxypiperidin-4-yl)methyl)-4-oxo-3,4-dihydropyrrolo[2,1-f][1,2,4]triazin-6-yl)-2,5-dimethylbenzamide;
N-(3-((1-(cyclopropanecarbonyl)-4-hydroxypiperidin-4-yl)methyl)-4-oxo-3,4-dihydropyrrolo[2,1-f][1,2,4]triazin-6-yl)-2-(trifluoromethoxy)benzamide;
2,4-dichloro-N-(3-((1-(cyclopropanecarbonyl)-4-hydroxypiperidin-4-yl)methyl)-4-oxo-3,4-dihydropyrrolo[2,1-f][1,2,4]triazin-6-yl)benzamide;
N-(3-((1-(cyclopropanecarbonyl)-4-hydroxypiperidin-4-yl)methyl)-4-oxo-3,4-dihydropyrrolo[2,1-f][1,2,4]triazin-6-yl)-2-phenylthiazole-4-carboxamide;
3-(4-chlorophenyl)-N-(3-((1-(cyclopropanecarbonyl)-4-hydroxypiperidin-4-yl)methyl)-4-oxo-3,4-dihydropyrrolo[2,1-f][1,2,4]triazin-6-yl)butanamide;
N-(4′-(4-hydroxy-4-((4-oxopyrrolo[2,1-f][1,2,4]triazin-3 (4H)-yl)methyl)piperidine-1-carbonyl)-[1,1′-biphenyl]-2-yl)methacrylamide;
3-((1-benzoyl-4-hydroxypiperidin-4-yl)methyl)-7-phenylimidazo[5,1-f][1,2,4]triazin-4(3H)-one;
3-((1-(4-fluorobenzoyl)-4-hydroxypiperidin-4-yl)methyl)-7-(4-fluorophenyl)imidazo[5,1-J][1,2,4]triazin-4(3H)-one;
3-((1-(4-fluorobenzoyl)-4-hydroxypiperidin-4-yl)methyl)-7-p-tolylimidazo[1,5-f][1,2,4]triazin-4 (3H)-one;
3-([4-Hydroxy-1-[3-(1H-pyrazol-1-yl)butanoyl]piperidin-4-yl]methyl)-7-(4-methylphenyl)-3H,4H-imidazo[4,34][1,2,4]triazin-4-one;
(S)-3-((1-(3-(1H-pyrazol-1-yl)butanoyl)-4-hydroxypiperidin-4-yl)methyl)-7-(p-tolyl) imidazo[5,1-f][1,2,4]triazin-4(3H)-one;
(R)-3-((1-(3-(1H-pyrazol-1-yl)butanoyl)-4-hydroxypiperidin-4-yl)methyl)-7-(p-tolyl) imidazo[5,1-f][1,2,4]triazin-4(3H)-one;
3-((1-(2-cyclopropyloxazole-5-carbonyl)-4-hydroxypiperidin-4-yl)methyl)-7-(4-fluorophenyl) imidazo[5,1-f][1,2,4]triazin-4(3H)-one;
3-((1-(4-(1H-pyrazol-1-yl)benzoyl)-4-hydroxypiperidin-4-yl)methyl)-7-(4-fluorophenyl) imidazo[5,1-f][1,2,4]triazin-4(3H)-one;
7-(4-fluorophenyl)-3-((4-hydroxy-1-(4-methylbenzoyl)piperidin-4-yl)methyl)imidazo[5,1-f][1,2,4]triazin-4(3H)-one; or
3-((1-(3-fluoro-4-methylbenzoyl)-4-hydroxypiperidin-4-yl)methyl)-7-(4-fluorophenyl) imidazo[5,1-f][1,2,4]triazin-4(3H)-one,
   and pharmaceutically acceptable salt thereof.

In some embodiments, the USP7 inhibitor is selected from compounds described in WO2013030218. In particular, the compound is selected from the group consisting of:
3-({4-hydroxy-1-[3-(2-methoxyphenyl)propanoyl]piperidin-4-yl}methyl)-3,4-dihydroquinazolin-4-one
3-({1-[2-(3-fluorophenoxy)acetyl]-4-hydroxypiperidin-4-yl}methyl)-3,4-dihydroquinazolin-4-one
3-{[4-hydroxy-1-(2-methylpropanoyl)piperidin-4-yl]methyl}-6,7-dimethoxy-3,4-dihydroquinazolin-4-one
3-{[4-hydroxy-1-(2-methylpropanoyl)piperidin-4-yl]methyl}-3,4-dihydroquinazolin-4-one
4-hydroxy-1-([2-methyl-3-(thiophen-2-yl)propanoyl]piperidin-4-yl}methyl)-3,4-dihydroquinazolin-4-one
7-chloro-3-{[1-(3-cyclopentylpropanoyl)-4-hydroxypiperidin-4-yl]methyl}-3,4-dihydroquinazolin-4-one
3-{[1-(3-cyclopentylpropanoyl)-4-hydroxypiperidin-4-yl]methyl}-3,4-dihydroquinazolin-4-one
7-chloro-3-{[4-hydroxy-1-(3-phenylpropanoyl)piperidin-4-yl]methyl}-3,4-dihydroquinazoli 4-one
3-{[4-hydroxy-1-(3-phenylpropanoyl)piperidin-4-yl]methyl}-3,4-dihydroquinazolin-4-one
7-chloro-3-({4-hydroxy-1-[2-methyl-3-(thiophen-2-yl)propanoyl]piperidin-4-yl}methyl)-3,4-dihydroquinazolin-4-one
3-({[4-hydroxy-1-(thiophen-2-yl)propanoyl]piperidin-4-yl}methyl)-3,4-dihydroquinazolin-4-one, and
3-{[1-(2-benzylpropanoyl)-4-hydroxypiperidin-4-yl]methyl}-3,4-dihydroquinazolin-4-one
3-{[1-(2-benzylpropanoyl)-4-hydroxypiperidin-4-yl]methyl}-7-chloro-3,4-dihydroquinazolin-4-one
   and pharmaceutically acceptable salt thereof.

In some embodiments, the USP7 inhibitor is selected from compounds described in WO2010081783. In particular, the compound is selected from the group consisting of:
4-acetyl-5-(3,4-di chloro-phenyl)-3-hydroxy-1-phenethyl-1,5-dihydro-pyrrol-2-one
4-acetyl-3-hydroxy-1-phenethyl-5-(4-cyanophenyl)-1,5-dihydro-2H-pyrrol-2-one
4-acetyl-3-hydroxy-1-phenethyl-5-(4-acetoxyphenyl)-1,5-dihydro-2H-pyrrol-2-one
4-acetyl-3-hydroxy-1-phenethyl-5-(4-thfluoromethoxyphenyl)-1,5-dihydro-2H-pyrrol-2-one
4-acetyl-3-hydroxy-1-phenethyl-5-(4-difluoromethoxyphenyl)-1,5-dihydro-2H-pyrrol-2-one
4-acetyl-3-hydroxy-1-phenethyl-5-(4-butoxyphenyl)-1,5-dihydro-2H-pyrrol-2-one
4-Acetyl-3-hydroxy-1-phenethyl-5-(4-thfluoromethyl-phenyl)-1,5-dihydro-pyrrol-2-one
4-acetyl-3-hydroxy-1-phenethyl-5-(4-n-octyloxyphenyl)-1,5-dihydro-2H-pyrrol-2-one
4-acetyl-5-[4-(3-dimethylaminopropoxy)-phenyl]-3-hydroxy-1-phenethyl-1,5-dihydro-2H-pyrrol-2-one
4-acetyl-3-hydroxy-1-phenethyl-5-(4-morpholinylphenyl)-1, 5-dihydro-2H-pyrrol-2-one
4-acetyl-5-(4-bromo-phenyl)-3-hydroxy-1-(2-pyri di n-4-yl-ethyl)-1,5-dihydro-pyrrol-2-one
4-acetyl-3-hydroxy-1-phenethyl-5-[4-(1H-tetrazol-5-yl)-phenyl]-1,5-dihydro-2H-pyrrol-2-one
4-acetyl-3-hydroxy-5-(4-isopropoxyphenyl)-1-phenethyl-1,5-dihydro-2H-pyrrol-2-one
4-acetyl-5-(4-bromo-phenyl)-3-hydroxy-1-(2-thiophen-2-yl-ethyl)-1, 5-dihydro-pyrrol-2-one
4-acetyl-5-(4-bromo-2-fluoro-phenyl)-3-hydroxy-1-phenethyl-1,5-dihydro-2H-pyrrol-2-one
4-acetyl-1-(2-biphenyl-4-yl-ethyl)-5-(4-bromo-phenyl)-3-hydroxy-1, 5-dihydro-pyrrol-2-one
4-(3-acetyl-4-hydroxy-5-oxo-1-phenethyl-2,5-dihydro-1H-pyrrol-2-yl)-benzoic acid 4-acetyl-3-hydroxy-5-(4-methane sulfonyl-phenyl)-1-phenethyl-1, 5-dihydro-2H-pyrrol-2-one
4-acetyl-3-hydroxy-1-phenethyl-5-pyridin-2-yl-1,5-dihydro-2H-pyrrol-2-one
4-acetyl-3-hydroxy-1-phenethyl-5-(4-hydroxy-phenyl)-1,5-dihydro-2H-pyrrol-2-one
4-acetyl-5-(4-tert-butyl-phenyl)-3-hydroxy-1-phenethyl-1,5-dihydro-2H-pyrrol-2-one
4-acetyl-5-(4-bromo-phenyl)-1-[2-(4-chloro-phenyl)-ethyl]-3-hydroxy-1,5-dihydro-pyrrol-2-one
4-acetyl-5-(4-bromo-phenyl)-1-[2-(4-bromo-phenyl)-ethyl]-3-hydroxy-1,5-dihydro-pyrrol-2-one
4-acetyl-5-biphenyl-4-yl-3-hydroxy-1-phen ethyl-1,5-dihydro-2H-pyrrol-2-one
4-acetyl-5-(4-bromo-phenyl)-1-[2-(3,4-dichloro-phenyl)-ethyl]-3-hydroxy-1,5-dihydro-pyrrol-2-one
5-(4-bromo-phenyl)-4-cyclopropanecarbonyl-3-hydroxy-1-phenethyl-1, 5-dihydro-pyrrol-2-one
4-acetyl-5-(4-bromo-phenyl)-3-hydroxy-1-[2-(3-methoxy-phenyl)-ethyl]-1, 5-dihydro-pyrrol-2-one
4-acetyl-5-(4-bromo-phenyl)-3-hydroxy-1-[2-(2-methoxy-phenyl)-ethyl]-1,5-dihydro-pyrrol-2-one
4-acetyl-5-(4-bromo-phenyl)-3-hydroxy-1-[2-(4-methoxy-phenyl)-ethyl]-1, 5-dihydro-pyrrol-2-one
4-acetyl-5-(4-bromo-phenyl)-3-hydroxy-1-[2-(4-fluorophenyl)-ethyl]-1, 5-dihydro-pyrrol-2-one
4-acetyl-5-(4-bromo-phenyl)-3-hydroxy-1-[2-(4-eth oxy-phenyl)-ethyl]-1, 5-dihydro-pyrrol-2-one
4-acetyl-3-hydroxy-1-phenethyl-5-pyridin-4-yl-1,5-dihydro-pyrrol-2-one
N-[4-(3-acetyl-4-hydroxy-5-oxo-1-phenethyl-2,5-dihydro-1H-pyrrol-2-yl)-phenyl]-acetamide
4-acetyl-5-benzo[1,3]dioxol-5-yl-3-hydroxy-1-phenethyl-1,5-dihydro-pyrrol-2-one
4-acetyl-3-hydroxy-1-phenethyl-5-pyridin-3-yl-1,5-dihydro-pyrrol-2-one
4-acetyl-5-benzo[1,3]dioxol-5-yl-1-[2-(3,4-dimethoxy-phenyl)-ethyl]-3-hydroxy-1,5-dihydro-pyrrol-2-one
4-acetyl-5-(4-chloro-3-fluoro-phenyl)-3-hydroxy-1-phenethyl-1,5-dihydro-pyrrol-2-one
4-acetyl-5-(3,4-dibromo-phenyl)-3-hydroxy-1-phenethyl-1,5-dihydro-pyrrol-2-one
4-acetyl-5-(2-chloro-pyridin-3-yl)-3-hydroxy-1-phenethyl-1,5-dihydro-pyrrol-2-one
4-acetyl-3-hydroxy-1-phenethyl-5-(6-trifluoromethyl-pyri din-3-yl)-1,5-dihydro-pyrrol-2-one
4-Acetyl-5-(4-bromo-phenyl)-1-[2-(2-chloro-phenyl)-ethyl]-3-hydroxy-1,5-dihydro-pyrrol-2-one
4-Acetyl-5-(4-chlorophenyl)-1-[2-(4-bromophenyl)-ethyl]-3-hydroxy-1,5-dihydro-pyrrol-2-one
4-Acetyl-5-(4-chlorophenyl)-1-[2-(4-chlorophenyl)-ethyl]-3-hydroxy-1,5-dihydro-pyrrol-2-one
4-Acetyl-5-(4-bromophenyl)-1-[2-(2,4-di chlorophenyl)-ethyl]-3-hydroxy-1,5-dihydro-pyrrol-2-one
4-acetyl-5-(4-bromo-3-chloro-phenyl)-3-hydroxy-1-phenethyl-1,5-dihydro-pyrrol-2-one
4-acetyl-3-hydroxy-5-(3-hydroxy-4-methoxy-phenyl)-1-phenethyl-1,5-dihydro-pyrrol-2-one
4-acetyl-1-(2-benzo[1,3]dioxol-5-yl-ethyl)-5-(4-chloro-phenyl)-3-hydroxy-1,5-dihydro-pyrrol-2-one
4-acetyl-5-(4-bromo-phenyl)-3-hydroxy-1-[2-(3-trifluoromethyl-phenyl)-ethyl]-1,5-dihydro-pyrrol-2-one
4-acetyl-5-(4-bromo-phenyl)-3-hydroxy-1-indan-2-yl-1,5-dihydro-pyrrol-2-one
4-acetyl-5-(4-bromophenyl)-1-[2-(3-bromophenyl)-ethyl]-3-hydroxy-1,5-dihydro-pyrrol-2-one
4-acetyl-5-(4-bromophenyl)-3-hydroxy-1-(2-m-tolyl-ethyl)-1,5-dihydro-pyrrol-2-one
4-Acetyl-5-(4-bromo-phenyl)-3-hydroxy-1-[2-(4-morpholin-4-yl-phenyl)-ethyl]-1,5-dihydro-pyrrol-2-one
4-acetyl-5-(4-bromophenyl)-3-hydroxy-1-(2-naphthalen-1-yl-ethyl)-1,5-dihydro-pyrrol-2-one
   and pharmaceutically acceptable salts.

In some embodiments, the USP7 inhibitor is P22077 (1-[5-[(2,4-Difluorophenyl)thio]-4-nitro-2-thienyl]-ethanone) as described in Altun M, Kramer H B, Willems L I, McDermott J L, Leach C A, Goldenberg S J et al. Activity-based chemical proteomics accelerates inhibitor development for deubiquitylating enzymes. Chem Biol 2011; 18: 1401-1412.

In some embodiments, the USP7 inhibitor is an inhibitor of USP7 expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of USP7 mRNA by binding thereto and thus preventing protein translation or increasing mRNA degradation, thus decreasing the level of USP7, and thus activity, in a cell. For example, antisense oligonucleotides of at least about 15 bases and complementary to unique regions of the mRNA transcript sequence encoding USP7 can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. USP7 gene expression can be reduced by contacting a subject or cell with a small double stranded RNA (dsRNA), or a vector or construct causing the production of a small double stranded RNA, such that USP7 gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing USP7. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

By a “therapeutically effective amount” is meant a sufficient amount of the USP7 inhibitor at a reasonable benefit/risk ratio applicable to the medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically, the USP7 inhibitor is combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Sterile injectable solutions are prepared by incorporating the active ingredient at the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The invention will be further illustrated by the following figures and examples. However, these examples and figures should not be interpreted in any way as limiting the scope of the present invention.

### FIGURES

FIG. 1: USP7 and CHK1 protein expression are correlated in AML cell lines and primary samples. A: CHK1 and USP7 protein levels were determined by western blot in different leukemic cell lines. Relative CHK1 and USP7 protein levels were normalized to actin loading control. HL-60 leukemic cell line served as internal reference and was set to 1. B: Linear regression analysis for the correlation between CHK1 and USP7 protein levels in leukemic cell lines. C: CHK1 and USP7 protein levels were determined by immunoblot in 57 primary AML samples. Actin was used as loading control. Samples are considered “high CHK1 abundance” if the average protein abundance value is higher than the median. Representative western blot of the 57 AML samples. D: Linear regression analysis for the correlation between CHK1 and USP7 protein levels in 21 primary AML samples with high CHK1 abundance.

FIG. 2: USP7 inhibition decreases CHK1 protein level. A: HL-60 cells were transfected with control (CTL) or USP7 (sequence #1) siRNA and protein levels were analyzed 48 h later by western blot with the indicated antibodies. Actin was used as loading control. Normalization was performed using CTL siRNA condition as reference. Data show means+/−s.e.m. from 6 independent experiments. For statistical analyses, nonparametric t-test was used. **P≤0.01, ****P≤0.0001. B: HL-60 cells were treated with USP7 inhibitor (P22077) at 10 and harvested at the indicated times, followed by western blotting for CHK1. Graph shows mean+/−s.e.m. of the quantification of CHK1 protein levels from 3 independent experiments in HL-60 cells normalized to actin loading control for the same condition. The amount of CHK1 at t=0 h was set to 1. For statistical analyses, t-test was used. *P≤0.05 and **P≤0.01. C: OCI-AML3 cells were treated with USP7 inhibitor (P22077) at 10 μM and harvested at the indicated times, followed by western blotting for CHK1 protein. Graph represent the quantification of CHK1 protein levels normalized to actin loading control from 3 independent experiments. Statistical analyses were performed as in B.

FIG. 3: USP7 interacts with CHK1 in AML cells and deubiquitinates CHK1 in leukemic cell lines. A: HL-60 whole-cell lysates were immunoprecipitated with anti-CHK1 antibody or an irrelevant Immunoglobulin (IgG) and immunoblotted with antibodies against the indicated proteins. Supernatant fraction is presented as SN. B: Similar experiment to A was performed with anti-CHK1 antibody from HEL cells transfected for 24 h with control (CTL) or CHK1 siRNA. C: Quantification of foci per cell was performed with ZEN and ImageJ software. For co-staining of CHK1 and USP7: mean=3.75+/−0.22 foci/cell from 1180 nuclei analyzed in siRNA control condition (n=4), mean=1.85+/−0.12 foci/cell from 1126 nuclei analyzed in siRNA CHK1 condition (n=4), and mean=2.69+/−0.21 foci/cell from 650 nuclei analyzed in siRNA USP7 condition (n=3). P-values were determined using the Mann-Whitney test, with ****P≤0.0001.

FIG. 4: USP7 inhibition impacts leukemic cells proliferation and viability in vitro and in vivo, without impacting normal cells. A: HL-60 cells were transfected with control (CTL) or USP7 siRNA for 48 h, then cell number was assessed by Trypan Blue staining. For statistical analyses, nonparametric t-test was used. *P≤0.05 and **P≤0.01. n=5. B: In similar experiments to the data in A, cell death was assessed by flow cytometry using annexinV/cell viability staining, using a MAC SQuant VYB flow cytometer and raw data were analyzed with FlowJo software. Data represent the mean+/−s.e.m. of 4 experiments. Statistical significance was proceeded by student's unpaired t-test. C: In similar experiments as described in A, cell cycle distribution was determined using propidium iodide (PI) staining and analyzed by flow cytometry using a MACSQuant VYB flow cytometer and completed by analyses with FlowJo software. Representative cell cycle distribution profiles on 3 independent experiments was shown. D: HL-60 cells were treated has indicated and, 48 h after, cell number was assessed by trypan blue. Statistical analyses were performed as in A (n=5). E: In similar experiments to the data in D, cell death was assessed by flow cytometry using annexinV/cell viability staining. Statistical significance was done as in B, (n=3). F: In similar experiments to the data in D, cell cycle distribution was determined using propidium iodide (PI) staining. Representative cell cycle distribution profiles on 3 independent experiments was shown. G: OCI-AML3 cells were treated has indicated and, 48 h after, cell number was assessed by Trypan Blue staining. Statistical analyses were performed as in A (n=6). H: In similar experiments to the data in G, cell death was assessed by flow cytometry using annexinV/cell viability staining. Statistical significance was done as in B, (n=5). I: In similar experiments to the data in G, cell cycle distribution was determined using propidium iodide (PI) staining. Representative cell cycle distribution profiles on 3 independent experiments was shown. J: Peripheral Blood Mononuclear Cells (PBMC, n=3) and AML primary samples (n=3) were treated with 10 μM P22077 for 24 h, and cell viability was assessed by flow cytometry using annexinV/cell viability staining, using a MACSQuant VYB flow cytometer and data were analyzed with FlowJo software. K: KaplanMeier curves of mice survival were established NSG mice engrafted with OCI-AML3, and treated with P22077 (30 mg/kg/day) or vehicle (10% DMSO in corn oil) during 5 days. For statistical analysis, Mantel-Cox test was used. ***P≤0.001.

FIG. 5: USP7 inhibition potentiates cytarabine treatment in AML A: Quantification of PLA foci from 3 independent experiments was performed with ZEN and ImageJ software. For co-staining of CHK1 and USP7: mean=1.07+/−0.07 foci/cell from 677 nuclei analyzed for untreated condition (NT); mean=3.31+/−0.21 foci/cell from 743 nuclei analyzed for AraC treatment; mean=0.68+/−0.04 foci/cell from 604 nuclei analyzed for P22077 treatment and mean=1.24+/−0.10 foci/cell from 407 nuclei analyzed for combined treatment. P-values were determined using the unpaired t-test, P≤0.0001****. B: HL-60 cells were treated as indicated for 24 h. Cell death was assessed by flow cytometry with annexinV/cell viability staining using a MACSQuant VYB flow cytometer and FlowJo software. Statistical analysis was performed by unpaired t-test (n=4). *P≤0.05 and **P≤0.01. C: OCI-AML3 cells were treated with P22077 (10 μM), AraC (1 μM) or the combination of both drugs, for 24 h. Cell death was assessed as in C. Statistics were established by unpaired t-test (n=3). *P≤0.05, **P≤0.01 and ns=not significant. D-E: Analyses of the clonogenic properties of high CHK1 abundance (high CHK1, n=6 primary samples, left panel) and low CHK1 abundance (low CHK1, n=3 primary samples, right panel) AML blast cells upon continuous exposure to 10 nM cytarabine (Ara-C) alone or in combination with 5 μM USP7 inhibitor (P22077). Colony formation was assessed after 7 days and represented as the ratio of the number of clones between untreated and treated conditions. For statistical analyses, unpaired Wilcoxon test was used. *P≤0.05, and ns=not significant.

### EXAMPLE

Methods:

Cell Lines, AML Samples and Treatments

Human leukemic cells lines were cultured as described in supplemental methods. Thawed samples (or derivative products, such as DNA and RNA) from 57 AML patients were analyzed for CHEK1 mRNA and CHK1 protein abundance after informed consent in accordance with the Declaration of Helsinki. The samples were stored at the HIMIP collection (BB-0033-00060). In conformance with French law, the HIMIP collection was declared to the Ministry of Higher Education and Research (DC 2008-307 collectionl) and obtained by transfer agreement (AC 2008-129) after approbation by ethical committees (Comite de Protection des Personnes Sud-Ouest et Outremer II and APHP ethical committee). Clinical and biological annotations of the samples have been declared to the CNIL (Comite National Informatique et Libertes).

The USP7 inhibitor, P22077 was purchased from Selleck Chemicals (S7133, Selleckchem, Houston, USA) and stored in DMSO at 10 mM. CHK1 inhibitor SCH900776 was purchased from Clinisciences (CliniSciences, Nanterre, France). TUH Pharmacy (Toulouse, France) was kind enough to provide us with Cytarabine (AraC).

Tumor Xenografts into NOD SCID Gamma (NSG) Mice

NOD/LtSz-SCID/IL-2Ry chain null (NSG) mice were bred at the UMS006 in Toulouse (France) using breeders obtained from Charles River. All animal experimental protocols were approved by the institutional Animal Care and Use Ethical Committee of the UMS006 and the Région Midi-Pyrénées (approval 2017071314596526). NSG mice were treated by i.p. injection of busulfan (20 mg/kg) on the day before the experiment. Mice were engrafted by injection of 2.10⁶ OCI-AML3 cells into the tail vein. Twenty-two days after injection, mice were randomly split into 4 groups: one group was treated with 100 μL corn oil with 10% DMSO and 100 PBS as vehicle (n=7), one group with 10 mg/kg of AraC in 100 μL PBS and 100 μL corn oil with 10% DMSO (n=7), one group was treated with 30 mg/kg of P22077 diluted in 100 corn oil with 10% DMSO (n=8), and one group was treated with the combination of the two drugs (n=8). Mice were treated by daily intraperitoneal (IP) injection with AraC, P22077 or vehicle for 5 days. Overall mouse survival was established under these conditions.

RNA Sequence Analysis

Single Cell Transcriptomics.

Blood cells of one patient at diagnosis and at his relapse after his chemotherapeutic induction were collected. AML cells were purified by ficoll centrifugation and then AML blast cells were sorted based on the expression of CD45+ and CD33+ and ANEXIN-. 500 cells per condition were used to performed a 10×genomics single cells assay as recommend by 10×Genomics. Sequencing was performed using an Illumina High seq 3000. Clusterization of the raw data was performed using 10×genomics cellranger pipeline.

Transcriptomic Signatures and Datamining.

Two USP7 signatures were generated from transcriptomes of AML patients with high versus low expression of USP7 from two independent databases (TCGA Network, 2013⁴⁰, and Verhaak data base GSE6891⁴¹). Gene Set Enrichment Analysis (GSEA) using USP7 signature was performed from the relapses and diagnosis transcriptomes of Hackl and al⁴² and the high and low responder in mice from Farge and al⁴³ and the LSC and bulk transcriptomes from Eppert and al⁴⁴. The analysis was performed using the GSEA3.0 software from the Broad Institute.

Results

USP7 and CHK1 Protein Expression are Correlated in AML

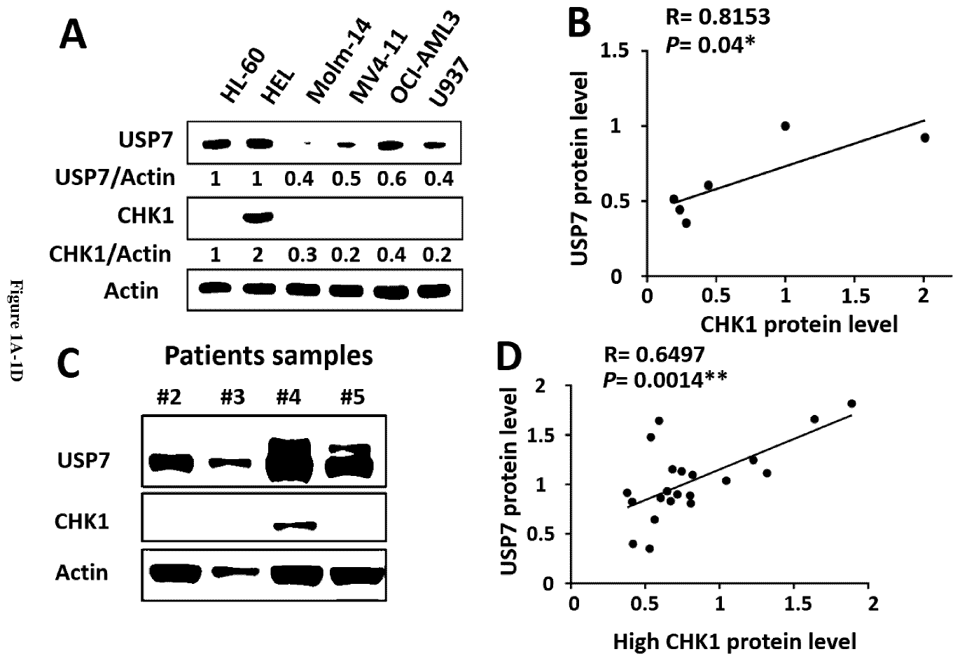

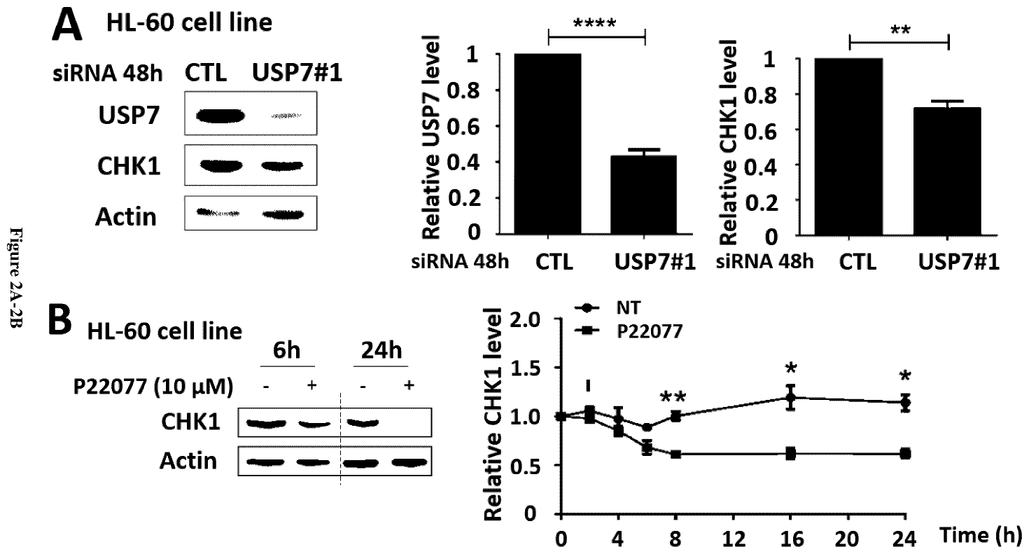

We first investigated CHK1 and USP7 protein levels in a panel of 6 AML cell lines by western blot analysis (FIG. 1A). Both CHK1 and USP7 protein expression levels were heterogeneous in leukemic cell lines, and were significantly correlated (FIG. 1B). We then performed a similar analysis in a cohort of primary AML samples. Out of 57 samples, 10 were not usable (degraded or not enough actin signal). Therefore, we performed the analysis on 47 patient samples. Based on the median expression of CHK1 protein, we separated this cohort into two groups of high CHK1 (#4 samples, FIG. 1C) and low CHK1 (#5 samples, FIG. 1C) expressing samples. As shown in the four examples of FIG. 1C, USP7 protein level is highly variable in AML. A significant correlation between CHK1 and USP7 protein levels was found in high CHK1 samples (FIG. 1D) but not in low CHK1 ones (data not shown). Altogether, these data strongly suggest a functional link between USP7 and CHK1 in leukemic cells expressing high CHK1 levels.

USP7 Promotes CHK1 Stabilization.

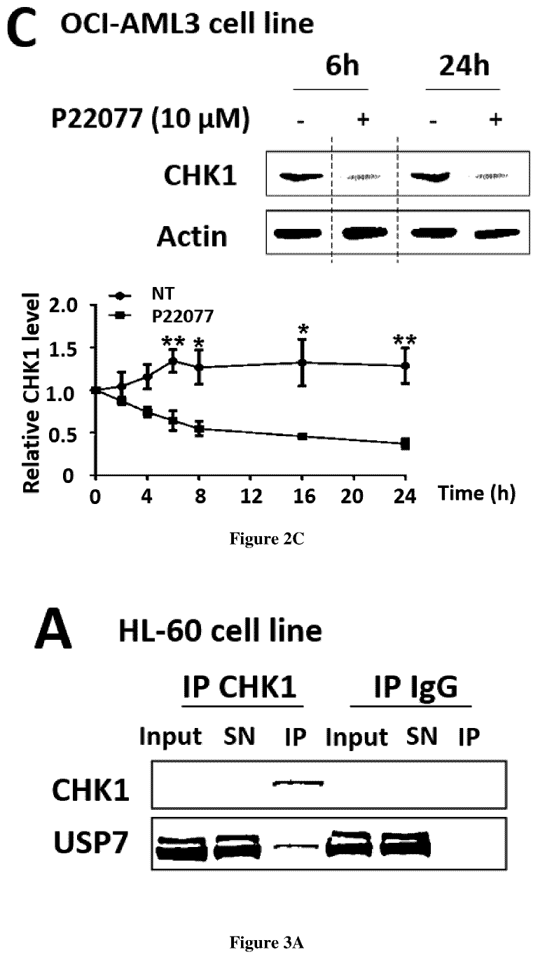

We then investigated whether USP7 could regulate CHK1 levels in leukemic cells. For this, we used the HL-60 cell line, which expresses high levels of CHK1 and USP7 proteins, and has a p53 null status (data not shown), avoiding the potential effects of p53 regulation by USP7.¹⁴⁻¹⁷ First, we silenced USP7 expression by RNA interference in this cell line, and we observed a significant decrease of CHK1 protein levels (FIG. 2A), although only half of USP7 protein was down regulated with this siRNA. Similar results were obtained with a second USP7 siRNA (data not shown). We then used P22077, an inhibitor of USP7 catalytic activity^{34,35}, to further monitor the impact of USP7 inhibition on CHK1 levels. P22077 treatment resulted in a significant decrease of CHK1 protein levels that was visible from 8 h to 24 h of treatment (FIG. 2B). We then asked whether p53 status could affect this response and reproduced these experiments on two AML cell lines, OCI-AML3 and HEL, which express wild-type p53 and high CHK1 protein levels (FIG. 1A and data not shown). As shown in FIG. 2C, P22077 treatment reduced CHK1 protein level in OCI-AML3 cell line in a similar way as in HL-60. USP7 silencing or inhibition with P22077 in HEL cells similarly decreased CHK1 protein levels (data not shown), without significant impact on p53 protein levels (data not shown). Using an immunofluorescence approach, we also validated that approximately 50% of CHK1 protein signal disappears after 24 hours of USP7 inhibition (data not shown). Together, these experiments show that genetic or pharmacological inhibition of USP7 decreases CHK1 protein levels in three different leukemic cell lines, independently of p53 status.

USP7 Interacts with CHK1 and Regulates its Deubiquitylation in AML Cells.

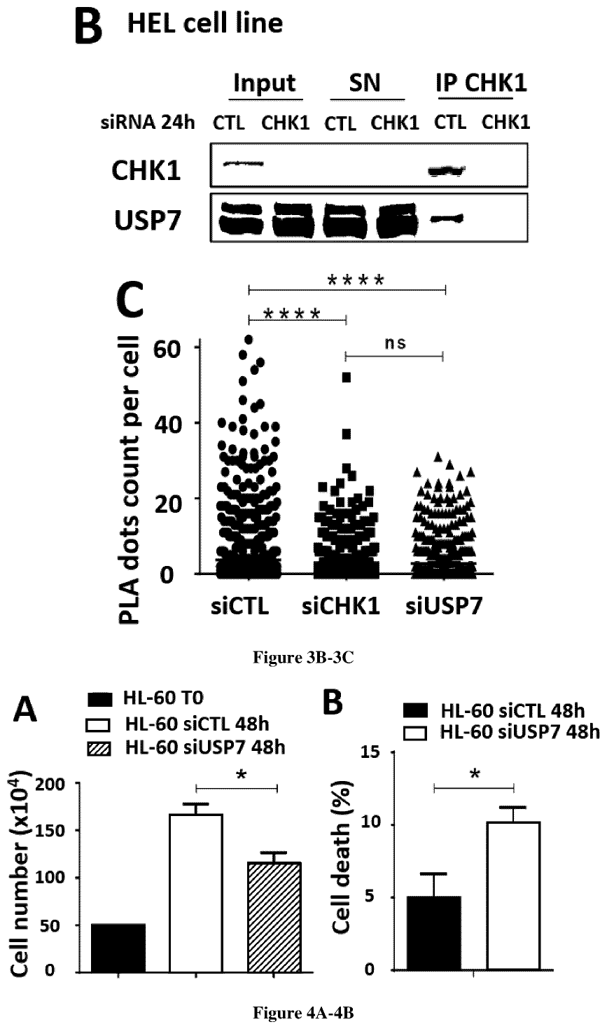

To determine whether USP7 and CHK1 interact in leukemic cells, we first performed co-immunoprecipitations of CHK1 from HL-60 and HEL cells (FIG. 3A-3B, respectively). In both cells lines, USP7 co-immunoprecipitated with CHK1, demonstrating the presence of the two proteins in the same complex.

This interaction was further confirmed by Proximity Ligation Assay (PLA) that allows to visualize proximity (30-40 nm) between two proteins. Through this approach, we confirmed that CHK1 and USP7 are co-localized in HL-60 cells, as shown by the presence of PLA dots in most cells (data not shown). The absence of staining in presence of a single PLA antibody was confirmed as recommended (data not shown). Moreover, CHK1 or USP7 silencing using siRNA significantly reduced the number of PLA dots, demonstrating the specificity of this interaction (FIG. 3C).

To confirm that USP7 can regulates CHK1 protein level, endogenous CHK1 was immunoprecipitated from HL60 cells treated or not with P22077, in presence of the proteasome inhibitor MG132 to stabilize ubiquitinated forms of the protein. Immunoblotting of these fractions was performed and probed with an antibody against ubiquitin. The results shown in (data not shown) indicate that CHK1 was more abundantly ubiquitinated following USP7 inhibition compared to the control condition, indicating that USP7 is a negative regulator of CHK1 ubiquitination.

To confirm this observation, we reproduced these experiments by overexpressing HA-tagged ubiquitin in HL60 cells, and by using siRNA against CHK1 or USP7. Endogenous CHK1 was then immunoprecipitated and immunoblotted with CHK1 and HA-antibodies. HA-ubiquitin signal present in CHK1 immunoprecipitates was higher when USP7 was silenced (data not shown), confirming the data obtained with USP7 inhibitor. Collectively, these results indicate that USP7 is an important mediator of CHK1 deubiquitination in leukemic cells.

We then investigated whether USP7 can regulate CHK1 protein level or its subcellular localization in leukemic cells. Having performed USP7 pharmacological inhibition in HL-60 (p53-) and OCI-AML3 (p53+) (data not shown), we went further and silenced USP7 expression by RNA interference in HL-60 and HEL (p53+) cell lines, and observed a significant decrease of CHK1 protein levels (data not shown), although only half of the USP7 proteins were down regulated with these two siRNA sequences in HL-60. Using immunofluorescence, we also validated that approximately 50% of the CHK1 protein signal disappears after 24 hours of USP7 inhibition, without any changes in CHK1 subcellular localization (data not shown). Together, these experiments show that genetic or pharmacological inhibition of USP7 decreases CHK1 protein levels in three different leukemic cell lines, independently of p53 status, demonstrating the role of USP7 deubiquitinase activity on CHK1 stability.

To complete, experiments were performed to test whether the half-life of CHK1 is regulated by USP7 in leukemic cells. To this end, HL60 cells were treated with P22077 and the half-life of CHK1 was measured in the presence of 50 μg/mL cycloheximide (CHX) to inhibit protein biogenesis. USP7 inhibition significantly shortened the half-life of CHK1 protein after 6 hours of P22077 treatment, without affecting CHEK1 mRNA expression (data not shown).

Finally, to further understand which poly-ubiquitin chain on CHK1 is removed by USP7, we immunoprecipitated equal amount of K48 linked poly-ubiquitin or K63 linked poly-ubiquitin proteins with specific antibodies in HL60 cells. The results indicated that K48-linked ubiquitin species conjugated on CHK1 were the major form of ubiquitin moieties (data not shown) and opposed by USP7 through its deubiquitinase activity (data not shown) in leukemic cells. Our data demonstrate that USP7 stabilizes CHK1 by preventing its proteasomal degradation/removing the K48-linked poly-ubiquitin.

Taken together, our results predict that targeting USP7 in leukemic cells, which exhibit a strong dependence on CHK1 level for their proliferation, can impair their survival.

USP7 Inhibition Impacts AML Cells Viability without Affecting Normal Cells.

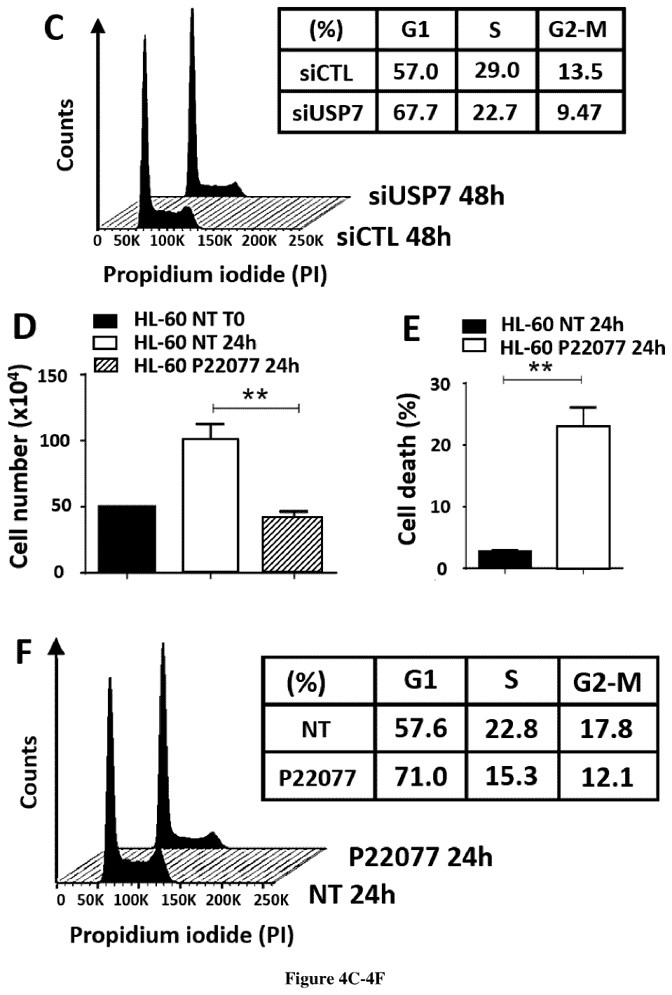

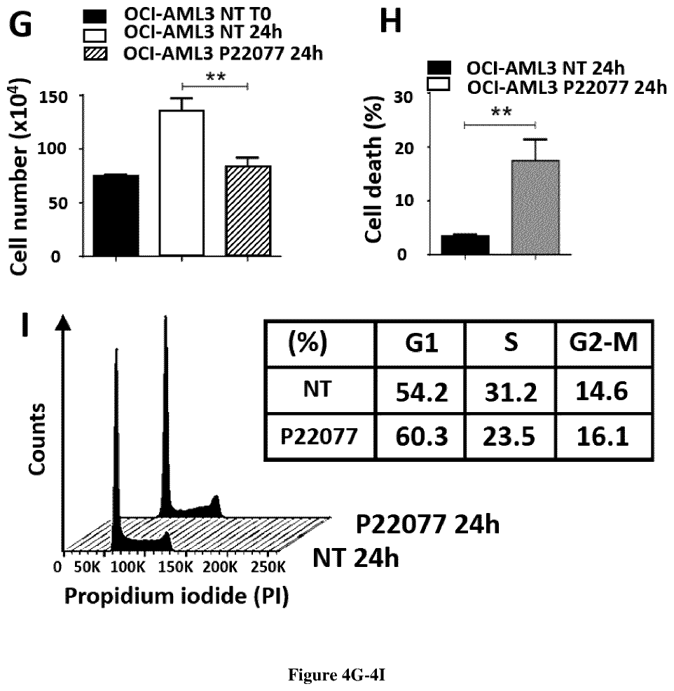

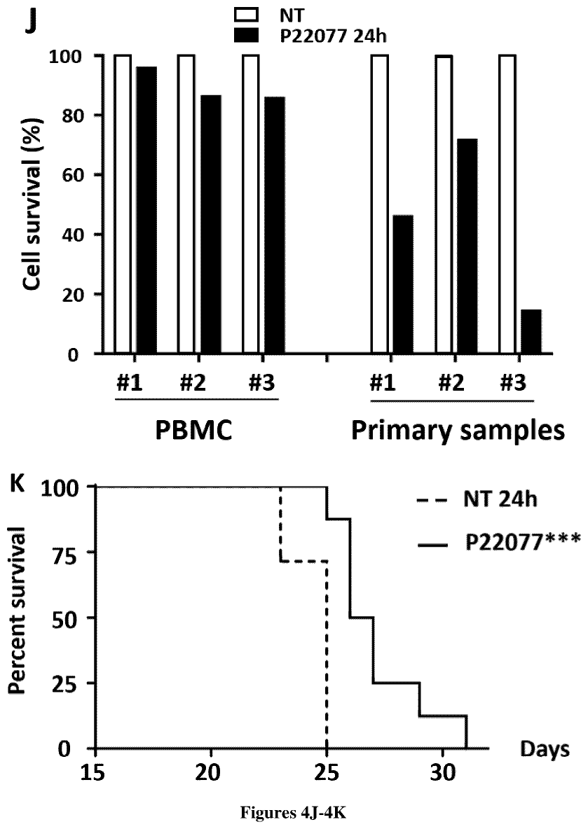

We then investigated the impact of USP7 silencing or pharmacological inhibition on the proliferation of leukemic cell lines. As shown in FIG. 4A, USP7 silencing in HL-60 cells significantly reduced cell proliferation, by increasing apoptotic cell death (FIG. 4B) and the proportion of cells in G1 phase (FIG. 4C).

Similar results were obtained by USP7 pharmacological inhibition in HL-60 and OCI-AML3 cell lines (FIGS. 4D-F and 4G-I, respectively), which indicates that the p53 pathway is not involved in the effect caused by USP7 inhibition or silencing.

We then performed similar experiments on primary AML cells and normal peripheral blood mononuclear cells (PBMC). As shown in FIG. 4J, USP7 inhibition with P22077 significantly reduced cell viability in three different primary AML samples, while PBMCs were largely unaffected by the treatment. Of note, primary AML and PBMCs classically do not proliferate in these culture conditions. Altogether, these results suggest that USP7 controls leukemic cells viability without affecting normal hematopoietic cell survival.

Finally, the effect of USP7 inhibition in vivo was studied in an AML xenograft model. OCI-AML3 resistant cells were injected into the tail vein of immunodeficient NSG mice to establish AML disease. USP7 inhibitor treatment significantly improved overall mouse survival (***p=0.0001) compared to vehicle treated mice (FIG. 4K).All these findings suggest that USP7 inhibition could have a therapeutic interest in AML.

USP7-CHK1 Proximity is Enhanced in Response to Cytarabine Treatment.

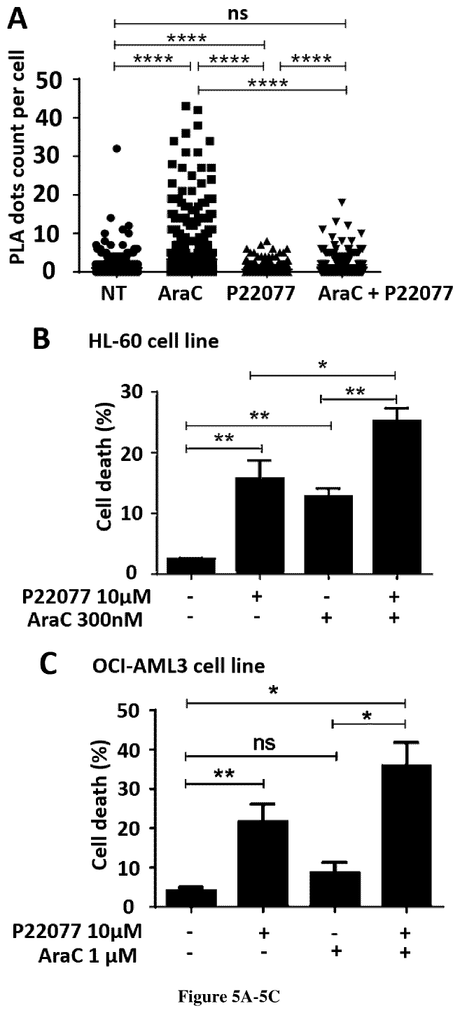

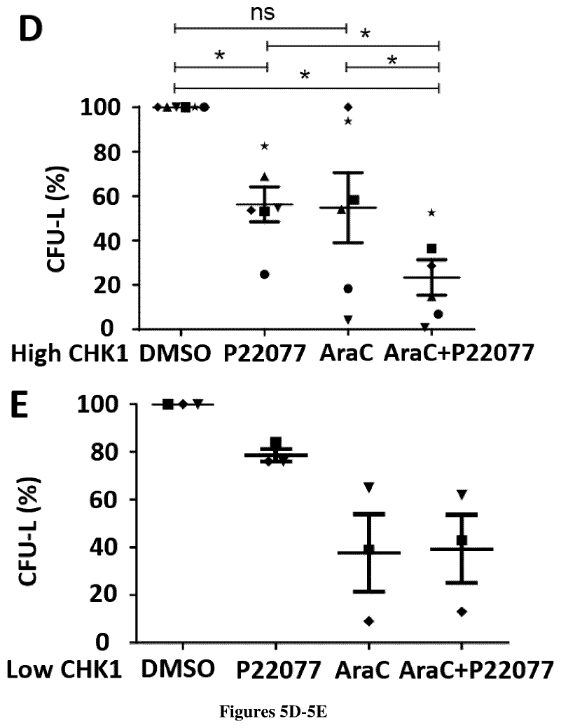

Given the importance of CHK1 in the resistance to genotoxic therapeutic drugs in AML⁶, USP7 could be a regulator of CHK1 in this context. To test this hypothesis, we monitored the CHK1/USP7 association by PLA in AML cells treated with cytarabine (AraC). A significant proximity between the two proteins was visualized in cytarabine-treated HL60 cells (FIG. 5A). The specificity of the interaction was confirmed by using USP7 inhibitor (P22077) that induced significant decrease in CHK1 protein level (FIG. 2B), dramatically reducing the number of PLA dots per cell (FIG. 5A). Similar results were obtained with the combination of cytarabine and P22077. Altogether, our data suggest a role of USP7 in promoting CHK1 stability in response to DNA damaging treatment.

Targeting USP7 Overcomes Cytarabine Resistance in AML.

To explore the possibility that USP7 by modulating CHK1 stability USP7 participates in the chemoresistance of leukemic cells, we examined the capacity of USP7 inhibition to sensitize HL60 and OCI-AML3 cells to cytarabine treatment. As shown in FIGS. 5B and 5C, P22077 synergistically enhances cell death induced by cytarabine in these resistant cell lines, independently of p53 status. We then quantified the capacity of primary AML cells to form colonies in methylcellulose-based semi-solid medium, when exposed to clinical relevant concentrations (10 nM) of cytarabine combined with the P22077 inhibitor (FIG. 5D-5E). Leukemic cells with high CHK1 levels (FIG. 5D) were significantly more resistant to cytarabine compared to cells expressing low amounts of CHK1 (FIG. 5E), which is consistent with our previous observations.⁶ it was also strikingly to note that USP7 inhibition potentiated the effects of cytarabine in cells expressing high levels of CHK1, leading to reduced colonies formation, while on the contrary, it did not modify the sensitivity to cytarabine of cells that expressed low amounts of CHK1. Our results indicate that P22077 significantly enhances the cytotoxic effect of cytarabine on leukemic cells by reducing their colony-forming potential. Since these data suggest that CHK1 is an important mediator of this effect, we measured the induction of cell death in response to a CHK1 inhibitor (SCH900776) in combination with or without cytarabine treatment (data not shown). These experiments showed that the P22077-induced cell death was mainly mediated by the USP7-CHK1 axis. Collectively, these results indicate that P22077 could represent an interesting anti-leukemic drug to override chemoresistance, in part due to its CHK1 destabilization capacity. This is consistent with our previous results highlighting the importance of this kinase in AML cells resistance to chemotherapy.

Transcriptomic Gene Signature and AML Patients Outcome.

Given our results and that USP7 has been implicated in transcription regulation,^{22,25,27} our next step was to consider whether cytarabine-resistant AML cells display a specific USP7-related transcriptomic signature. To test this, USP7 abundance was quantified in AML patient samples. We found that USP7 transcript abundance and USP7 protein levels are highly correlated (R=0.8073, **p=0.0085) (data not shown). We then defined a USP7 specific gene signature (41 genes upregulated, data not shown) from The Cancer Genome Atlas transcriptomic database⁴⁰, and found that this signature is highly enriched in the transcriptome of AML, patient samples at relapse (HACKL cohort. GEO: GSE6891⁴²) (data not shown). We also observed a similar enrichment in transcriptomes of cytarabine-resistant human AML cells purified from Patient Derived Xenograft (PDX) models treated with clinically relevant doses of cytarabine (GEO: GSE97631⁴³) (data not shown). These data highlight a distinct correlation between high USP7 transcriptomic signature and AML cell resistance to chemotherapeutic drugs. Therefore, USP7 signature could represent a new predictive marker of chemoresistance in AML.

To further characterize primary AML samples heterogeneity, we performed single cell RNA sequencing of AML cells collected either at diagnosis or at relapse from an AML patient (IM10) treated with combination of anthracyclin and cytarabine. Based on their different gene expression, we identified two different transcriptional clusters of cells at diagnosis and relapse (data not shown), but at relapse, cluster 2 was strikingly decreased compared to cluster 1 (data not shown). The signature of cluster 1 corresponds to the gene signature of the chemoresistant cells in this patient. Interrogation of two publicly available transcriptomic datasets established from AML patients at diagnosis (TCGA Network, 2013⁴⁰, and Verhaak data base GSE6891⁴¹) revealed that this chemoresistant gene signature was enriched in AML samples with high USP7 abundance (data not shown). Finally, we assessed the USP7 and CHK1 protein abundance in primary AML samples and found that IM10 leukemic primary sample at diagnosis presented high levels of both proteins (data not shown) which is consistent with the data described above. Altogether, these data strongly suggest that high USP7 levels are associated with chemoresistance in AML cells, and with the initiation of relapse following standard chemotherapeutic treatment.

Discussion

In this study, we identified USP7 as a key regulator of CHK1 levels in leukemic cells and revealed that USP7 plays an important role in AML chemoresistance.

While we previously documented the heterogeneity of CHK1 abundance in primary AML samples, the mechanism by which CHK1 mRNA and protein expressions are controlled in leukemic cells remained to be defined.⁶ These present data indicate that USP7 is one of the important regulators of CHK1 protein levels in AML cells and that this regulation governs the response of AML cells to genotoxic stress. In a recent study, we documented that high CHK1 protein levels favor cellular resistance to cytarabine, in part by facilitating fork progression or stalled fork restart.⁶ Our results suggest that high CHK1 levels, stabilized by USP7, allows cells to survive by enabling them to adapt to DNA replication stress induced by cytarabine treatment. In this context, cells with high USP7 expression may survive to the selective pressure of cytarabine treatment and become enriched, forming a residual aggressive tumor burden at the origin of relapse. This hypothesis is supported by our transcriptomic analyses showing that USP7 transcriptomic signature is enriched at relapse as well as in chemoresistant cells.

Recent studies reported that USP7 level is elevated in several types of tumors, and that USP7 overexpression is often predictive of a poor prognosis.^{37,45,46,47} Several studies consequently proposed USP7 as an attractive pharmaceutical target for various cancers, and pharmacological inhibitors of this enzyme have been recently developed.

In this study, we observed heterogeneous USP7 levels in cell lines and primary samples and established that patients with a high USP7 signature were more prone to chemoresistance and relapse. Experiments using single cell analysis support these findings by showing that the resistant gene signature is enriched in AML, samples that exhibit high USP7 levels, which reveals that this chemoresistant cell population pre-exist at diagnosis and is enriched following first line treatment. Consequently, these data suggest that USP7 could be a new prognostic marker and a potential therapeutic target for AML. Our finding that USP7 inhibition in vitro and in vivo significantly suppressed leukemic cell growth, alone or in combination with chemotherapy, independently of p53 status supports this notion. Although, p53 can be stabilized by USP7¹⁴⁻¹⁷, it appears that in AML, which does not frequently present p53 mutations, cells do not rely on p53 stabilization. Despite the large spectrum of USP7 substrates, we showed that the cytotoxic effect caused by USP7 inhibition is mediated, at least in part, by decreasing CHK1 function due to the fact that we observed similar anti-leukemic effect with the CHK1 inhibitor SCH900776. Therefore, we believe that dysregulation of a USP7-CHK1 axis may represent a new “Achilles heel” in AML cells. In fact, USP7 inhibition has a profound impact on proliferation and viability in leukemic cells expressing high CHK1 levels compared to low CHK1 expressing cells or peripheral blood mononuclear cells. This characteristic could provide an interesting therapeutic window in which this class of inhibitor could be proposed for clinical trials. It is interesting to note that our data are comparable to results observed in T-cell acute lymphoblastic leukemia. ⁴⁸

It will be important to investigate the mechanisms controlling USP7 expression in the context of AML heterogeneity. In AML samples, we established that USP7 transcript and protein abundance are well correlated, suggesting that USP7 might be mostly regulated at the transcriptional level. Previous reports have documented the transcriptional regulation of USP7, for instance in T-cell acute lymphoblastic leukemia (T-ALL) in which the transcription factor NOTCH1 induces USP7 gene expression, and where USP7 controls the stability of NOTCH1 and the JMJD3 histone demethylase through a positive regulatory loop.^{48,49} It is noteworthy that the NOTCH signaling pathway has been reported to play oncogenic and tumor suppressor functions in the hematopoietic system, although the biological and clinical relevance remains unclear in AML.⁵⁰ potential regulatory and functional link between USP7 and NOTCH1 is supported by our transcriptomic analyses since Delta Like non-canonical Notch ligand 1 (DLK1) was upregulated in cells enriched in USP7 gene signature. DKLJ encodes the NOTCH activator Delta1 which is involved in primary and model AML cell growth.⁵¹ Furthermore, previous analyses showed a significantly shorter survival of patients with high NOTCH1 or Delta1 expression, suggesting that activation of the NOTCH pathway may be associated with poor prognosis in AML.⁵² All these studies combined with our data, strongly suggest an interesting NOTCH/USP7/CHK1 axis involved in AML chemoresistance that should be investigated in future studies.

Finally, the large spectrum of USP7 substrates and its implication in fundamental cellular and developmental processes define USP7 as a molecule of great importance. Depending on its targeted substrates and cellular context, USP7 may be either a tumor suppressive and oncogenic protein. Consequently, it will be important to understand how the acute leukemic cells highjack USP7 functions.

In summary, our study provide evidence that USP7 regulates CHK1 abundance and stability in chemoresistant AML cells. Moreover, the combination of USP7 inhibitor with cytarabine induces synergistic anti-leukemic activity. Overall, our study further confirms that targeting USP7 is a promising therapeutic strategy to treat Acute Leukemia and supports the inclusion of USP7 inhibitors into clinical studies aimed at overcoming chemoresistance in AML.

### REFERENCES

Throughout this application, various references describe the state of the art to which this invention pertains. The disclosures of these references are hereby incorporated by reference into the present disclosure.
1. Döhner H, Weisdorf D J, Bloomfield C D. Acute myeloid leukemia. N Engl J Med. 2015; 373: 1136-1152.
2. Papaemmanuil E, Gerstung M, Bullinger L, et al. Genomic Classification and Prognosis in Acute Myeloid Leukemia. N Engl J Med. 2016; 374(23):2209-2221.
3. Yang M, Zhao J, Liu T, et al. Use of FLT3 inhibitors in acute myeloid leukemia remission induction or salvage therapy: systematic review and meta-analysis. Cancer Manag Res. 2018; 10:2635-2652.
4. DiNardo C D, Stein E M, de Botton S, et al. Durable Remissions with Ivosidenib in IDH1-Mutated Relapsed or Refractory AML. N Engl J Med. 2018; 378(25):2386-2398.
5. Zhou J, Chng W J. Resistance to FLT3 inhibitors in acute myeloid leukemia: Molecular mechanisms and resensitizing strategies. World J Clin Oncol. 2018; 9(5):90-97.
6. David L, Fernandez-Vidal A, Bertoli S, et al. CHK1 as a therapeutic target to bypass chemoresistance in AML. Sci Signal. 2016; 9(445).
7. Zhang Y W, Otterness D M, Chiang G G et al. Genotoxic stress targets human Chk1 for degradation by the ubiquitin-proteasome pathway. Mol Cell. 2005; 19:607-618.
8. Huh J, Piwnica-Worms H. CRL4CDT2 targets CHK1 for PCNA-independdent destruction. Mol Cell Biol. 2013; 33:213-226.
9. Leung-Pineda V, Huh J, Piwnica-Worms H. DDB1 targets Chk1 to the Cul4 E3 ligase complex in normal cycling cells and ini cells experiencing replication stress. Cancer res. 2009; 69:2630-2637.
10. Zhang Y W, Brognard J, Coughlin C, et al. The F Box Protein Fbx6 regulates Chk1 srability and cellular sensitivity to replication stress. Mol Cell. 2009; 35:442-453.
11. Guervilly J H, Renaud E, Takata M, Rosselli F. USP1 deubiquitinase maintains phosphorylated CHK1 by limiting its DDB1-dependent degradation. Hum Mol Genet. 2011; 20(11):2171-81.
12. Cheng Y C, Shieh S Y. Deubiquitinating enzyme USP3 controls CHK1 chromatin association and activation. PNAS. 2018; 115(21):5546-51.
13. Alonsa de Vega I, Martin Y, Smits V A J. USP7 controls Chk1 protein stability by direct deubiquitination. Cel Cycle. 2014; 13:3921-3926.
14. Tavana O, Gu W. Modulation of the p53/MDM2 interplay by HAUSP inhibitors. Journal of Mol Cell Bio. 2017; 9(1):45-52.
15. Li M, Brooks C L, Kon N, Gu W. A dynamic role of HAUSP in the p53-Mdm2 pathway. Mol Cell. 2004; 13(6):879-886
16. Brooks C L, Gu W. p53 ubiquitination: Mdm2 and beyond. Mol Cell. 2006; 21(3):307-315
17. Mungamuri S K, Qiao R F, Yao S, Manfredi J J, Gu W, Aaronson S A. USP7 Enforces Heterochromatinization of p53 Target Promoters by Protecting SUV39H1 from MDM2-Mediated Degradation. Cell Rep. 2016; 14(11):2528-37.
18. Basu B, Saha G, Choudhury S G, Ghosh M K. Cellular homeostasis or tumorigenesis: USP7 playing the double agent. Cancer Cell and MicroEnv.2017; 4
19. van der Knaap J A, Kumar B R, Moshkin Y M. GlVIP synthetase stimulates histone H2B deubiquitylation by the epigenetic silencer USP7. Mol Cell. 2005; 17(5):695-707.
20. Faustrup H, Bekker-Jensen S, Bartek J, Lukas J, Mailand N. USP7 counteracts SCFbetaTrCP-but not APCCdh1-mediated proteolysis of Claspin. J Cell Biol. 2009; 184(1):13-9.
21. Du Z, Song J, Wang Y, et al. DNMT1 stability is regulated by proteins coordinating deubiquitination and acetylation-driven ubiquitination. Sci Signal. 2010; 3(146):ra80.
22. Zhu Q, Sharma N, He J, Wani G, Wani A A. USP7 deubiquitinase promotes ubiquitin-dependent DNA damage signaling by stabilizing RNF168. Cell Cycle. 2015; 14(9):1413-25.
23. Van der Horst A, de Vries-Smits A M, Brenkman A B, et al. FOXO4 transcriptional activity is regulated by monoubiquitination and USP7/HAUSP. Nat Cell Biol. 2006; 8(10):1064-73.
24. Song M S, Salmena L, Carracedo A, et al. The deubiquitinylation and localization of PTEN are regulated by a HAUSP-PML network. Nature. 2008; 455(7214):813-7.
25. Van der Knaap J A, Kozhevnikova E, Langenberg K, Moshkin Y M, Verrijzer C P. Biosynthetic enzyme GMP synthetase cooperates with ubiquitin-specific protease 7 in transcriptional regulation of ecdysteroid target genes. Mol Cell Biol. 2010; 30(3):736-44.
26. Holowaty M N, Frappier L. HAUSP/USP7 as an Epstein-Barr virus target. Biochem Soc Trans. 2004; 32(Pt 5):731-2.
27. Du Z, Song J, Wang Y, et al. DNMT1 stability is regulated by proteins coordinating deubiquitination and acetylation-driven ubiquitination. Sci Signal. 2010; 3(146):ra80.
28. Everett R D., Meredith M., On A., Cross A., Kathoria M., Parkinson J. A novel ubiquitin-specific protease is dynamically associated with the PML nuclear domain and binds to a herpesvirus regulatory protein. EMBO J. 1997; 16:1519-1530.
29. Nicholson B, Marblestone J G, Butt T R, Mattern M R. Deubiquitinating enzymes as novel anticancer targets. Future Oncol. 2007; 3(2):191-9.
30. D'Arcy P, Wang X, Linder S. Deubiquitinase inhibition as a cancer therapeutic strategy. Pharmacol Ther. 2015; 147:32-54
31. Kategaya L, Di Lello P, Rouge L, et al. USP7 small-molecule inhibitors interfere with ubiquitin binding. Nature. 2017; 550(7677):534-538.
32. Turnbull A P, Ioannidis S, Krajewski W W, et al. Molecular basis of USP7 inhibition by selective small-molecule inhibitors. Nature. 2017; 550(7677):481-486
33. Lamberto I, Liu X, Seo H S, et al. Structure-Guided Development of a Potent and Selective Non-covalent Active-Site Inhibitor of USP7. Cell Chem Biol. 2017; 24(12):1490-1500.
34. Pozhidaeva A, Valles G, Wang F, et al. USP7-Specific Inhibitors Target and Modify the Enzyme's Active Site via Distinct Chemical Mechanisms. Cell Chem Biol. 2017; 24(12):1501-1512
35. Desroses M, Altun M. The Next Step Forward in Ubiquitin-Specific Protease 7 Selective Inhibition. Cell Chem Biol. 2017 Dec. 21; 24(12):1429-1431.
36. Gavory G, O'Dowd C R, Helm M D, et al. Discovery and characterization of highly potent and selective allosteric USP7 inhibitors. Nat Chem Biol. 2018; 14(2):118-125.
37. Carra G, Panuzzo C, Torti D, et al. Therapeutic inhibition of USP7-PTEN network in chronic lymphocytic leukemia: a strategy to overcome TP53 mutated/deleted clones. Oncotarget. 2017; 8(22):35508-35522.
38. Chauhan D, Tian Z, Nicholson B, et al. A small molecule inhibitor of ubiquitin-specific protease-7 induces apoptosis in multiple myeloma cells and overcomes bortezomib resistance. Cancer Cell. 2012; 22(3):345-58.
39. Fan Y H, Cheng J, Vasudevan S A, et al. USP7 inhibitor P22077 inhibits neuroblastoma growth via inducing p53-mediated apoptosis. Cell Death Dis. 2013; 4:e867.
40. Network TCGAR. Genomic and epigenomic landscapes of adult de novo acute myeloid leukemia. N Engl J Med. 2013; 368:2059-74.
41. Verhaak R G W, Wouters B J, Erpelinck C A J, et al. Prediction of molecular subtypes in acute myeloid leukemia based on gene expression profiling. Haematologica. 2009; 94:131-4.
42. Hackl and al 2011 GEO: GSE6891
43. Farge T, Saland E, de Toni F, et al. Chemotherapy-Resistant Human Acute Myeloid Leukemia Cells Are Not Enriched for Leukemic Stem Cells but Require Oxidative Metabolism. Cancer Discov. 2017; 7(7):716-735.
44. Eppert K, Takenaka K, Lechman E R, et al. Stem cell gene expression programs influence clinical outcome in human leukemia. Nat Med. 2011; 17:1086-93. 45. An T, Gong Y, Li X, et al. USP7 inhibitor P5091 inhibits Wnt signaling and colorectal tumor growth. Biochem Pharmacol. 2017; 131:29-39.
46. Zhao G Y, Lin Z W, Lu C L, et al. USP7 overexpression predicts a poor prognosis in lung squamous cell carcinoma and large cell carcinoma. Tumour Biol. 2015; 36(3):1721-9.
47. Ma M, Yu N. Ubiquitin-specific protease 7 expression is a prognostic factor in epithelial ovarian cancer and correlates with lymph node metastasis. Onco Targets Ther. 2016; 9:1559-69.
48. Shan H, Li X, Xiao X, et al. USP7 deubiquitinates and stabilizes NOTCH1 in T-cell acute lymphoblastic leukemia. Signal Transduct Target Ther. 2018; 3:29.
49. Jin Q, Martinez C A, Arcipowski K M, et al. USP7 cooperates with NOTCH1 to drive the oncogenic transcriptional program in T cell leukemia. Clin Cancer Res. 2018 pii: clincanres.1740.2018.
50. Lobry C, Oh P, Mansour M R, Look A T, Aifantis I. Notch signaling: switching an oncogene to a tumor suppressor. Blood. 2014; 123(16):2451-9.
51. Wang S, Itoh M, Shiratori E, Ohtaka M, Tohda S. NOTCH activation promotes glycosyltransferase expression in human myeloid leukemia cells. Hematol Rep. 2018; 10(3):7576.
52. Xu X, Zhao Y, Xu M, et al. Activation of Notch signal pathway is associated with a poorer prognosis in acute myeloid leukemia. Med Oncol. 2011; 28 Suppl 1:S483-9.

1. A method of treating acute myeloid leukemia (AML) in a patient in need thereof comprising administering to the patient a therapeutically effective amount of a USP7 inhibitor.

2. The method of claim 1, wherein the AML is chemoresistant acute myeloid leukemia (AML).

3. The method of claim 1, further comprising administering to the patient a therapeutically effective amount of at least one chemotherapeutic agent and in combination with the USP7 inhibitor.

4. A method for enhancing the potency of a chemotherapeutic agent administered to a patient suffering from AML as part of a treatment regimen, the method comprising administering to the patient a pharmaceutically effective amount of a USP7 inhibitor in combination with at least one chemotherapeutic agent.

5. A method of preventing relapse in a patient suffering from AML who was treated with chemotherapy comprising administering to the patient a therapeutically effective amount of a USP7 inhibitor.

6. The method according to claim 3, wherein the chemotherapeutic agent is cytarabine (cytosine arabinoside, Ara-C, Cytosar-U), quizartinib (AC220), sorafenib (BAY 43-9006), lestaurtinib (CEP-701), midostaurin (PKC412), carboplatin, carmustine, chlorambucil, dacarbazine, ifosfamide, lomustine, mechlorethamine, procarbazine, pentostatin, (2′deoxycoformycin), etoposide, teniposide, topotecan, vinblastine, vincristine, paclitaxel, dexamethasone, methylprednisolone, prednisone, all-trans retinoic acid, arsenic trioxide, interferon-alpha, rituximab, gemtuzumab ozogamicin, imatinib mesylate, Cytosar-U), melphalan, busulfan, thiotepa, bleomycin, platinum (cisplatin), cyclophosphamide, daunorubicin, doxorubicin, idarubicin, mitoxantrone, 5-azacytidine, cladribine, fludarabine, hydroxyurea, 6-mercaptopurine, methotrexate, 6-thioguanine, and combinations thereof.

7. The method according to claim 3 wherein the chemotherapeutic agent is a combination of daunorubicin, or idarubicin plus cytarabine (AraC).

8. The method according to claim 3 wherein the chemotherapeutic agent is a BCL2 inhibitor.

9. The method according to claim 3 wherein the chemotherapeutic agent is a FLT3 inhibitor.

10. The method according to claim 3 wherein the chemotherapeutic agent is an IDH (isocitrate dehydrogenase) inhibitor.
